(19) United States
(12) Patent Application Publication
(10) Pub. No.: US 2016/0033532 A1
(43) Pub. Date: Feb. 4, 2016

(54) MAMMALIAN PROTEIN CO-RECOGNITION BY BROADLY NEUTRALIZING ANTIBODIES AS MODIFIED IMMUNOGENS FOR RE-ELICITATION

(71) Applicants: International AIDS Vaccine Initiative, New York, NY (US); The Scripps Research Institute, La Jolla, CA (US)

(72) Inventors: Richard Wyatt, La Jolla, CA (US); Karen Tran, La Jolla, CA (US); Javier Guenaga, La Jolla, CA (US); Shailendra Kumar, La Jolla, CA (US)

(21) Appl. No.: 14/813,519

(22) Filed: Jul. 30, 2015

Related U.S. Application Data
(60) Provisional application No. 62/032,484, filed on Aug. 1, 2014.

(51) Int. Cl. G01N 33/68; A61K 39/00

(52) U.S. Cl. CPC G01N 33/6878; A61K 39/0005; A61K 2039/57; A61K 2039/58; A61K 2039/645; G01N 2440/38; G01N 2333/47; G01N 2500/04

(57) ABSTRACT

The present invention relates to using HIV-1 broadly neutralizing antibodies to screen for glycan-dependent or protein-dependent self reactivities inherent in these mutated antibodies, and defining this cross recognition at the molecular level, and utilizing the information to re-elicit trimer-specific and/or N-glycan-dependent or protein-surface-dependent broadly neutralizing antibodies and therapeutic applications thereof.

### RELATED APPLICATIONS AND INCORPORATION BY REFERENCE

This application claims benefit of and priority to U.S. provisional patent application Ser. No. 62/032,484 filed Aug. 1, 2014.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted electronically in ASCII format and is hereby incorporated by reference in its entirety. Said ASCII copy, created on Aug. 19, 2015, is named 43094.01.2030_SL.txt and is 96,878 bytes in size.

### FIELD OF THE INVENTION

The present invention relates to screening for glycan-dependent or protein-dependent self reactivities, and defining this cross recognition at the molecular level, and utilizing the information to re-elicit trimer-specific and N-glycan-dependent or-protein-dependent broadly neutralizing antibodies and therapeutic applications thereof.

### BACKGROUND OF THE INVENTION

Most licensed antiviral human vaccines protect by neutralizing antibodies (NAbs) and passive administration of HIV-1 NAbs can protect non-human primates from viral challenge indicating that the elicitation of broadly NAbs (bNAbs) by vaccination can potentially prevent HIV infection.

The targets for HIV-1 NAbs, which exhibit varying degrees of breadth and potency, are the viral envelope glycoproteins (Env). The HIV-1 exterior envelope glycoprotein, gp120, and the transmembrane glycoprotein, gp41, are derived from the cleavage of gp160 precursor protein and are the only virally encoded proteins on the surface of the virus. These non-covalently associated glycoproteins form the trimeric functional spike on the virus surface and mediate viral entry into host cells. The gp120 subunit binds first to the primary receptor, CD4, and then engages the co-receptor, usually CCR5 as a second step in the entry process. The gp41 subunit then mediates virus-to-cell membrane fusion and entry of viral genomic information as two RNA copies into susceptible target cells.

Viral entry into cells can be blocked by elicited antibodies that can efficiently recognize the native functional spike. However, there are issues of antibody recognition that are inherent in the variable nature of Env. For example, host selection pressures have made most circulating isolates resistant to V3 ‘tip’ antibodies (since most individuals generate such responses, forcing viral escape and, although the virus may be sensitive to V1V2-directed antibodies, the high level of variation tolerated in these surface-exposed ‘loops’ often generates escape variants to these type-specific antibodies.

Historically, only four human monoclonal antibodies (mAbs) derived from HIV-1-infected individuals were identified that can neutralize a broad spectrum of primary isolates in vitro. Of these four mAbs, two are directed against gp120 epitopes and are known as 2G12 and b12. The other two mAbs, 2F5 and 4E10, recognize contiguous epitopes within the gp41 membrane proximal external region (MPER). All four of these bNAbs can protect against several routes of virus challenge after passive administration before, or immediately following, exposure to virus (SHIV). These results indicate that, under certain circumstances, NAbs alone can protect against acquisition of HIV infection.

In the past several years, there has been marked interest in defining the antibody specificities mediating neutralization breadth in the serum of HIV-1 infected individuals. Based on these studies, it is now clear that approximately 10-20% of chronically HIV-1 infected individuals develop serum neutralization breadth.

In some cases, the broad neutralizing activity elicited during natural HIV-1 infection can be mapped to sub-regions of Env, such as gp120. The first two bNAbs of the 2^{nd} generation of such mAbs, PG9 and PG16, were isolated from an HIV-1 infected individual by expansion of all B cells and screening. These related antibodies strongly prefer to recognize the gp120 Env subunit in the context of a trimer, bind to an epitope at the V region cap of Env (V2) and display both remarkable breadth and potency of neutralization as well as “self”, N-linked glycan recognition.

Soon after, the broad and potent CD4 binding site (CD4bs)-directed antibody, VRC01, was cloned from an HIV-1 infected individual whose serum possessed neutralization breadth that was previously mapped to the CD4 interactive region. This was done by flow cytometry-based sorting (FACS) using selective protein probes. A plethora of new bNAbs to the CD4bs mAbs have now been described, also isolated by FACS, using Env-based sorting of memory B cells; the so-called PGT mAbs that target glycan and conserved elements in V1V2 or the base of V3 at the top of the spike have also been described.

Another CD4bs-directed antibody, VRC03, was isolated from the same patient as VRC01, and a somewhat less broad CD4bs antibody. Since then, the related bNAbs VRC06 and VRC06b have also been isolated from this same patient. Another CD4bs HJ16, was isolated from a different patient by slightly different methodology. Other CD4bs-directed bNabs have been isolated (refs) and remarkably, several of these use the same VH gene (VH1-02), and mediate most recognition of Env by the germline encoded HCDR2 region.

In yet other studies, the putative Transmitter/founder (T/F) virus has been isolated as well as the putative un-mutated ancestral BCR as a mAb, which remarkably binds to the T/F Env. One of these bNAbs recognizes N-glycan as part of its epitope as do all of the so-called PGT bNAbs. In other broadly neutralizing patient sera, in rare instances, the specificity of the broad neutralizing activity can be mapped to the gp41 MPER region and a new and potent MPER mAb, 10E8, was isolated and now the trimer-specific bNAb PGT151 was reported.

Citation or identification of any document in this application is not an admission that such document is available as prior art to the present invention.

### SUMMARY OF THE INVENTION

Taken together, the isolation of this myriad of bNAbs from multiple infected donors demonstrates that, given the appropriate immunogen and immunization regimen, it may be possible to induce similar Abs by vaccination.

Thereby proof-of-principle induction of broadly neutralizing antibodies using any means is of high interest even if the real world application of such principle may face some future obstacles. This is akin to the malarial vaccine in which proof-of-principle and protective mechanism of the liver stage was demonstrated by isolation of sporozytes even if this approach may never be feasible as a real world vaccine. The malarial studies demonstrate feasibility and mechanisms of protection that may be accomplished by more practical future vaccine candidates, and the same rationale applies herein.

The innovation of this application is multi-fold, but relies predominantly on the identification of self-ligands recognized primarily by the N-glycan recognizing anti-HIV-1 bNAbs, including the highly potent PGT class of mAbs, but also may include the VRC CD4bs-directed mAbs. The major innovation is the concept, compelled by the emerging higher resolution definition of the native HIV Env spike, is that the highly glycosylated nature of the spike allows only rare mAbs to penetrate the glycan shield to access the underlying immunogenic polypeptide surface. It is now clear that the virus has “employed”, through host selective forces and evolution of course, N-glycan-mediated shielding to permit only small patches of the underlying protein surface to be accessible by protein interfacial probes such as antibodies, only in relatively to extremely rare circumstances and then with other constraints that make them problematic and improbable to elicit with a frequency within an individual or the human population sum to significantly impact on the persistent replication and transmission/dissemination of the virus within the human population. These other required bNAb properties, or constraints, are long HCDR3 loops that are selected against due to the increased frequency of self-reactivity and high level of somatic hypermutation (SHM) that then borders on violation of peripheral tolerance and SHM-driven auto-reactivity and finally, reactivity with host self-N-linked glycan. Similar principles have been established for the hydrophobic MPER, which is expanding to the CD4 binding site, but the glycan-reactive antibodies represent an extensive and relatively common class of bNAbs to apply innovative screening, mapping and immunogenicity in rationally based attempts to re-elicit such mAbs by defining the underlying genesis of their elicitation in the very HIV-infected individuals in which they occur.

Therefore, this invention relates to screening for such glycan-dependent self reactivities, revealing in preliminary data a strong recognition of PGT151 and its family members for an abundant glycoprotein galectin 3 binding protein (gal3BP), to then define this cross recognition at the molecular level, and to then use this novel and ground breaking information to re-elicit this trimer-specific and N-glycan-dependent bNAb. Applicants then propose to expand this screening, identification process and re-elicitation strategy to other putative bNAb; self-glycoprotein ligand:ligand pairs.

The invention relates to a ground breaking overarching principle that the recently described broadly neutralizing HIV-1 directed antibodies possess self-reactivity likely due to their unusual properties of long HCDR3s, glycan-reactivity and extremely and extraordinary levels of SHM.

The invention utilizes state-of-the-art molecular mapping and high resolution definition of binding requirements to determine if the glycan-recognizing, PGT bNAbs recognize putative ligands and do they use the very binding site that recognizes the HIV-1 Env trimer as well.

The invention utilizes novel approaches to both break tolerance and to combine usage of heterologous T cell help to show proof of principle re-elicitation, in a reproducible manner, using antibodies with broadly neutralizing properties and to then close the loop regarding the cross-reactivity of such elicited bNabs.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 shows PGT 151 immunoprecipitates (IPs) detect an unaccounted for band of apparent MW 90 kD from HEK 293F supernatants when low levels of Env trimer are co-expressed by these cells following transient HIV-1 Env SOSIP plasmid DNA transfection.

FIG. 2 shows a band excised for mass spec (MS) analysis immuno-precipitated (IP) by PGT 151 from “spent” 293F TC supernatants.

FIG. 3 shows PGT151 family member recognition of gal3BP and SOSIP. IPs of 293F spent media and media expressing clade C 16055 SOSIP by PGT151 and family members 151-3, 155-6 and 158 as shown. Note that PGT152 appears to better recognize SOSIP compares to Gal3BP whereas PGT155 better recognizes gal3BP relative to SOSIP, indicating potential cross-competition of these two glycoproteins for binding by PGT151.

FIG. 4 shows EM of gal3BP. And EM image is shown of gal3BP and PGT151 Fab. Relatively low concentrations of gal3BP are shown and the PGT 151 Fab is not clearly resolved in this image.

FIG. 5 shows octet binding curves of recombinant gal3BP in solution recognition by PGT151. Shown are the binding curves of selected N-glycan deletion mutants as described.

FIG. 6 shows binding of PGT151 to HIV-1 envelope glycoprotein SOSIP trimers and recombinant human G3BP expressed from 293F cells.

FIG. 7 shows that PGT151 readily binds to G3BP produced in 293F cells; however, reduced binding is seen with G3BP produced in 293 cells with Kifunensine (yields high mannose) or G3BP produced in 293S (GnT1−/−) cells which do not contain complex glycans, suggesting binding is mediated through complex glycans.

FIG. 8 shows that G3BP competes with JRFL SOSIP trimer for binding with PGT151.

FIG. 9 shows that all members of the PGT151 family binds to recombinant human G3BP with different affinities.

FIG. 10 shows that the PGT151 germ line (gL) nor the chimeric PGT151 mAb with germline heavy chain (gHC) and mature kappa light chain (KC) bind to G3BP. Modestly reduced binding is seen with the chimeric PGT151 mAb with mature heavy chain (HC) and germline light chain (gL KC), indicating binding is predominantly mediated through the mature HC. Significantly reduced binding to the SOSIP trimer is seen with germline versions of PGT151, indicating binding is predominantly mediated through both mature heavy and light chains.

FIG. 11 shows sera from wild type C57/b6 mice immunized with G3BP or G3BP with CpG cross reacts with the envelope core, V3S, as well as to gp140 foldon produced in 293F cells. Shown above, average IgG reactivity from each group (n=6) after each immunization (post 1-3) by ELISA. Shown below are the post 3 midpoint IgG titers.

FIG. 12 shows sera from wild type C57/b6 mice immunized with G3BP cross reacts with the envelope core, V3S, produced in 293F cells but not to the deglycosylated core, suggesting binding is mediated through the complex N glycans on the core surface.

FIG. 13 shows sera from rabbits immunized with different HIV envelope glycoproteins (i.e. 8b core, 3G hyperglycosylated core, gp140 foldon) produced in 293F cells cross reacts with recombinant human G3BP expressed from 293F cells.

FIG. 14 shows sera from guinea pigs immunized with different HIV-1 envelope glycoproteins (i.e. core followed by 1 or 2 SOSIP trimer boosts) produced in 293F cells cross reacts with recombinant human G3BP expressed from 293F cells.

FIG. 15 shows sera from NHPs (rhesus macaques) immunized with different HIV envelope glycoproteins (i.e. gp140 foldon of NFL trimer) produced in 293F cells cross reacts with recombinant human G3BP expressed from 293F cells.

### DETAILED DESCRIPTION OF THE INVENTION

The elicitation of broadly neutralizing antibodies to the HIV-1 envelope glycoprotein (Env) spike remains a critical goal of a broadly effective vaccine. A major obstacle in the elicitation of HIV-neutralizing antibodies is the extensive glycosylation of the Env, which creates a “self-appearing” and relatively occluding shield to most antibodies directed toward the underlying Env peptide surface elicited by infection or vaccination.

The recent identification of several new and novel glycan-related bNAbs isolated from infected-individuals targeting both gp120 and gp41 determinants of the spike are an extremely exciting and encouraging development, although they are relatively infrequent in occurrence. These bNAbs indicate that the human immune system can indeed generate broadly effective and glycan-dependent neutralizing responses. However, these bNAbs do possess some unusual properties, such as high levels of somatic hypermutation (SHM), long HCDR3s and self-glycan reactivity, properties that are not commonly found in the human naïve repertoire.

Here, Applicants propose to use these new broadly neutralizing antibody tools to interrogate mammalian glycoprotein repertoires to identify human or mammalian non-HIV self-antigens. The identification of mammalian non-HIV proteins may be valuable reagents to elicit bNAbs as proof-of-principle, once tolerance is broken to these self-antigens. This is especially true if some of the glycan-reactive bNAbs have been generated by normally anergic (naïve) B cells, tolerized to the very glycoproteins that may have been part of the antigenic drive to affinity mature the bNAbs. This may in part be due to the pathogenic conditions that exist, persist and may even worsen over the duration of chronic HIV infection; that is some loss of tolerance due to negative impacts on the regulatory CD4 T cell compartment, the major regulator of central B cell tolerance.

The present invention relates to galectin 3 binding protein (gal3BP) and variants thereof, such as fusion proteins to re-elicit PGT151-like bNAbs. Examples of gal3BP fusion proteins include, but are not limited to, Gal3BP-His for capture on liposomes for priming or boosting with similarly captured Env trimers, Gal3BP-PADRE for priming or boosting to break tolerance by providing heterologous T cell help to this mammalian self protein in combination with well ordered soluble gp140 Env trimers containing PADRE, Gal3BP-TT peptide for priming or boosting to break tolerance by providing heterologous T cell help to this mammalian self protein in combination with well-ordered soluble gp140 Env trimers containing TT peptide, Gal3BP-HA peptide for priming or boosting to break tolerance by providing heterologous T cell help to this mammalian self protein in combination with well-ordered soluble gp140 Env trimers containing HA peptide and Gal3BP- coupled to HPV L1 for priming or boosting to break tolerance by providing heterologous T cell help to this mammalian self protein in combination with well-ordered soluble gp140 Env trimers also coupled or not to HPV L1 particles. Other modifications include, but are not limited to, galectin 3 binding protein modified with N/C His, Padre, TT peptide (P30), and free Cysteine. For example, the His tag may be used to capture the gal3BP on liposomes or some other particle for multivariant array. Similarly for the free cysteine, to see if Applicants may biochemically array on HPV L1 protein or Q beta particles, etc. The PADRE and TT peptides may be used for heterologous T cell help to break tolerance and as well to link to the NFL or SOSIP trimers to provide common T cell helper peptides for prime:boosting with the HIV trimers.

Applicants have also seen that PGT121 recognizes Peroxidasin (PXDN), expressed at high levels in heart tissue and smooth muscle. Applicants have also observed that VRC06 recognizes Clathrin Heavy Chain 1 (CLTC), is involved in clatharin coated pits, and is widely expressed. The present invention also contemplates utilizing PXDN and CLTC in a matter similar to gal3bp.

The invention encompasses a method of eliciting trimer-specific and/or N-glycan-dependent broadly neutralizing antibodies in a patient in need thereof which may comprise administering a non-HIV protein of the present invention. The the non-HIV protein may be modified and/or arrayed on a particle.

The arraying on particle may be on an HPV particle or a liposome. Applicants append a free cysteine residue to the self-reactive protein and express and purify recombinant proteins possessing the free cysteine at the C- or N-termini to couple to recombinant HPV particles also engineered to possess free and arrayed cysteine residues on their molecular surface. Exemplary sequences for the cysteine (C) fusion proteins are provided in the present application.

Exemplary examples of modified Gal3BP binding proteins and sequences thereof are provided below.

Galectin 3 Binding Protein; Gal3BP; G3BP

1. Recombinant G3BP with His tag

>G3BP-His
(SEQ ID NO: 1)
MTPPRLFWVWLLVAGTQGVNDGDMRLADGGATNQGRVEIFYRGQWGTVCD
NLWDLTDASVVCRALGFENATQALGRAAFGQGSGPIMLDEVQCTGTEASL
ADCKSLGWLKSNCRHERDAGVVCTNETRSTHTLDLSRELSEALGQIFDSQ
RGCDLSISVNVQGEDALGFCGHTVILTANLEAQALWKEPGSNVTMSVDAE
CVPMVRDLLRYFYSRRIDITLSSVKCFHKLASAYGARQLQGYCASLFAIL
LPQDPSFQMPLDLYAYAVATGDALLEKLCLQFLAWNFEALTQAEAWPSVP
TDLLQLLLPRSDLAVPSELALLKAVDTWSWGERASHEEVEGLVEKIRFPM
MLPEELFELQFNLSLYWSHEALFQKKTLQALEFHTVPFQLLARYKGLNLT
EDTYKPRIYTSPTWSAFVTDSSWSARKSQLVYQSRRGPLVKYSSDYFQAP
SDYRYYPYQSFQTPQHPSFLFQDKRVSWSLVYLPTIQSCWNYGFSCSSDE
LPVLGLTKSGGSDRTIAYENKALMLCEGLFVADVTDFEGWKAAIPSALDT
NSSKSTSSFPCPAGHFNGFRTVIRPFYLTNSSGVDGGGGHHHHHH*
>His-G3BP
(SEQ ID NO: 2)
MTPPRLFWVWLLVAGTQGHHHHHHGGGGVNDGDMRLADGGATNQGRVEIF
YRGQWGTVCDNLWDLTDASVVCRALGFENATQALGRAAFGQGSGPIMLDE
VQCTGTEASLADCKSLGWLKSNCRHERDAGVVCTNETRSTHTLDLSRELS
EALGQIFDSQRGCDLSISVNVQGEDALGFCGHTVILTANLEAQALWKEPG
SNVTMSVDAECVPMVRDLLRYFYSRRIDITLSSVKCFHKLASAYGARQLQ
GYCASLFAILLPQDPSFQMPLDLYAYAVATGDALLEKLCLQFLAWNFEAL
TQAEAWPSVPTDLLQLLLPRSDLAVPSELALLKAVDTWSWGERASHEEVE
GLVEKIRFPMMLPEELFELQFNLSLYWSHEALFQKKTLQALEFHTVPFQL
LARYKGLNLTEDTYKPRIYTSPTWSAFVTDSSWSARKSQLVYQSRRGPLV
KYSSDYFQAPSDYRYYPYQSFQTPQHPSFLFQDKRVSWSLVYLPTIQSCW
NYGFSCSSDELPVLGLTKSGGSDRTIAYENKALMLCEGLFVADVTDFEGW
KAAIPSALDTNSSKSTSSFPCPAGHFNGFRTVIRPFYLTNSSGVDGGGGH
HHHHH*

2. Recombinant G3BP with PADRE

>His PADRE-G3BP
(SEQ ID NO: 3)
MTPPRLFWVWLLVAGTQGHHHHHHGGSGAKFVAAWTLKAAAGSGVNDGDM
RLADGGATNQGRVEIFYRGQWGTVCDNLWDLTDASVVCRALGFENATQAL
GRAAFGQGSGPIMLDEVQCTGTEASLADCKSLGWLKSNCRHERDAGVVCT
NETRSTHTLDLSRELSEALGQIFDSQRGCDLSISVNVQGEDALGFCGHTV
ILTANLEAQALWKEPGSNVTMSVDAECVPMVRDLLRYFYSRRIDITLSSV
KCFHKLASAYGARQLQGYCASLFAILLPQDPSFQMPLDLYAYAVATGDAL
LEKLCLQFLAWNFEALTQAEAWPSVPTDLLQLLLPRSDLAVPSELALLKA
VDTWSWGERASHEEVEGLVEKIRFPMMLPEELFELQFNLSLYWSHEALFQ
KKTLQALEFHTVPFQLLARYKGLNLTEDTYKPRIYTSPTWSAFVTDSSWS
ARKSQLVYQSRRGPLVKYSSDYFQAPSDYRYYPYQSFQTPQHPSFLFQDK
RVSWSLVYLPTIQSCWNYGFSCSSDELPVLGLTKSGGSDRTIAYENKALM
LCEGLFVADVTDFEGWKAAIPSALDTNSSKSTSSFPCPAGHFNGFRTVIR
PFYLTNSSGVD*
>PADRE-G3BP His
(SEQ ID NO: 4)
MTPPRLFWVWLLVAGTQGAKFVAAWTLKAAAGSGVNDGDMRLADGGATNQ
GRVEIFYRGQWGTVCDNLWDLTDASVVCRALGFENATQALGRAAFGQGSG
PIMLDEVQCTGTEASLADCKSLGWLKSNCRHERDAGVVCTNETRSTHTLD
LSRELSEALGQIFDSQRGCDLSISVNVQGEDALGFCGHTVILTANLEAQA
LWKEPGSNVTMSVDAECVPMVRDLLRYFYSRRIDITLSSVKCFHKLASAY
GARQLQGYCASLFAILLPQDPSFQMPLDLYAYAVATGDALLEKLCLQFLA
WNFEALTQAEAWPSVPTDLLQLLLPRSDLAVPSELALLKAVDTWSWGERA
SHEEVEGLVEKIRFPMMLPEELFELQFNLSLYWSHEALFQKKTLQALEFH
TVPFQLLARYKGLNLTEDTYKPRIYTSPTWSAFVTDSSWSARKSQLVYQS
RRGPLVKYSSDYFQAPSDYRYYPYQSFQTPQHPSFLFQDKRVSWSLVYLP
TIQSCWNYGFSCSSDELPVLGLTKSGGSDRTIAYENKALMLCEGLFVADV
TDFEGWKAAIPSALDTNSSKSTSSFPCPAGHFNGFRTVIRPFYLTNSSGV
DGGGGHHHHHH*
>G3BP-Padre His
(SEQ ID NO: 5)
MTPPRLFWVWLLVAGTQGVNDGDMRLADGGATNQGRVEIFYRGQWGTVCD
NLWDLTDASVVCRALGFENATQALGRAAFGQGSGPIMLDEVQCTGTEASL
ADCKSLGWLKSNCRHERDAGVVCTNETRSTHTLDLSRELSEALGQIFDSQ
RGCDLSISVNVQGEDALGFCGHTVILTANLEAQALWKEPGSNVTMSVDAE
CVPMVRDLLRYFYSRRIDITLSSVKCFHKLASAYGARQLQGYCASLFAIL
LPQDPSFQMPLDLYAYAVATGDALLEKLCLQFLAWNFEALTQAEAWPSVP
TDLLQLLLPRSDLAVPSELALLKAVDTWSWGERASHEEVEGLVEKIRFPM
MLPEELFELQFNLSLYWSHEALFQKKTLQALEFHTVPFQLLARYKGLNLT
EDTYKPRIYTSPTWSAFVTDSSWSARKSQLVYQSRRGPLVKYSSDYFQAP
SDYRYYPYQSFQTPQHPSFLFQDKRVSWSLVYLPTIQSCWNYGFSCSSDE
LPVLGLTKSGGSDRTIAYENKALMLCEGLFVADVTDFEGWKAAIPSALDT
NSSKSTSSFPCPAGHFNGFRTVIRPFYLTNSSGVDGSGAKFVAAWTLKAA
AGGGGHHHHHH*

3. Recombinant G3BP with TT (tetanus toxoid) peptide P30

>TT-P30 G3BP His
(SEQ ID NO: 6)
MTPPRLFWVWLLVAGTQGFNNFTVSFWLRVPKVSASHLEGSGVNDGDMRL
ADGGATNQGRVEIFYRGQWGTVCDNLWDLTDASVVCRALGFENATQALGR
AAFGQGSGPIMLDEVQCTGTEASLADCKSLGWLKSNCRHERDAGVVCTNE
TRSTHTLDLSRELSEALGQIFDSQRGCDLSISVNVQGEDALGFCGHTVIL
TANLEAQALWKEPGSNVTMSVDAECVPMVRDLLRYFYSRRIDITLSSVKC
FHKLASAYGARQLQGYCASLFAILLPQDPSFQMPLDLYAYAVATGDALLE
KLCLQFLAWNFEALTQAEAWPSVPTDLLQLLLPRSDLAVPSELALLKAVD
TWSWGERASHEEVEGLVEKIRFPMMLPEELFELQFNLSLYWSHEALFQKK
TLQALEFHTVPFQLLARYKGLNLTEDTYKPRIYTSPTWSAFVTDSSWSAR
KSQLVYQSRRGPLVKYSSDYFQAPSDYRYYPYQSFQTPQHPSFLFQDKRV
SWSLVYLPTIQSCWNYGFSCSSDELPVLGLTKSGGSDRTIAYENKALMLC
EGLFVADVTDFEGWKAAIPSALDTNSSKSTSSFPCPAGHFNGFRTVIRPF
YLTNSSGVDGGGGHHHHHH*
>His TT-P30 G3BP
(SEQ ID NO: 7)
MTPPRLFWVWLLVAGTQGHHHHHHGGSGFNNFTVSFWLRVPKVSASHLEG
SGVNDGDMRLADGGATNQGRVEIFYRGQWGTVCDNLWDLTDASVVCRALG
FENATQALGRAAFGQGSGPIMLDEVQCTGTEASLADCKSLGWLKSNCRHE
RDAGVVCTNETRSTHTLDLSRELSEALGQIFDSQRGCDLSISVNVQGEDA
LGFCGHTVILTANLEAQALWKEPGSNVTMSVDAECVPMVRDLLRYFYSRR
IDITLSSVKCFHKLASAYGARQLQGYCASLFAILLPQDPSFQMPLDLYAY
AVATGDALLEKLCLQFLAWNFEALTQAEAWPSVPTDLLQLLLPRSDLAVP
SELALLKAVDTWSWGERASHEEVEGLVEKIRFPMMLPEELFELQFNLSLY
WSHEALFQKKTLQALEFHTVPFQLLARYKGLNLTEDTYKPRIYTSPTWSA
FVTDSSWSARKSQLVYQSRRGPLVKYSSDYFQAPSDYRYYPYQSFQTPQH
PSFLFQDKRVSWSLVYLPTIQSCWNYGFSCSSDELPVLGLTKSGGSDRTI
AYENKALMLCEGLFVADVTDFEGWKAAIPSALDTNSSKSTSSFPCPAGHF
NGFRTVIRPFYLTNSSGVD*
>G3BP TT-P30 His
(SEQ ID NO: 8)
MTPPRLFWVWLLVAGTQGVNDGDMRLADGGATNQGRVEIFYRGQWGTVCD
NLWDLTDASVVCRALGFENATQALGRAAFGQGSGPIMLDEVQCTGTEASL
ADCKSLGWLKSNCRHERDAGVVCTNETRSTHTLDLSRELSEALGQIFDSQ
RGCDLSISVNVQGEDALGFCGHTVILTANLEAQALWKEPGSNVTMSVDAE
CVPMVRDLLRYFYSRRIDITLSSVKCFHKLASAYGARQLQGYCASLFAIL
LPQDPSFQMPLDLYAYAVATGDALLEKLCLQFLAWNFEALTQAEAWPSVP
TDLLQLLLPRSDLAVPSELALLKAVDTWSWGERASHEEVEGLVEKIRFPM
MLPEELFELQFNLSLYWSHEALFQKKTLQALEFHTVPFQLLARYKGLNLT
EDTYKPRIYTSPTWSAFVTDSSWSARKSQLVYQSRRGPLVKYSSDYFQAP
SDYRYYPYQSFQTPQHPSFLFQDKRVSWSLVYLPTIQSCWNYGFSCSSDE
LPVLGLTKSGGSDRTIAYENKALMLCEGLFVADVTDFEGWKAAIPSALDT
NSSKSTSSFPCPAGHFNGFRTVIRPFYLTNSSGVDGSGFNNFTVSFWLRV
PKVSASHLEGGGGHHHHHH*

4. Recombinant G3BP with His tag and free terminal Cys

>G3BP-His*C
(SEQ ID NO: 9)
MTPPRLFWVWLLVAGTQGVNDGDMRLADGGATNQGRVEIFYRGQWGTVCD
NLWDLTDASVVCRALGFENATQALGRAAFGQGSGPIMLDEVQCTGTEASL
ADCKSLGWLKSNCRHERDAGVVCTNETRSTHTLDLSRELSEALGQIFDSQ
RGCDLSISVNVQGEDALGFCGHTVILTANLEAQALWKEPGSNVTMSVDAE
CVPMVRDLLRYFYSRRIDITLSSVKCFHKLASAYGARQLQGYCASLFAIL
LPQDPSFQMPLDLYAYAVATGDALLEKLCLQFLAWNFEALTQAEAWPSVP
TDLLQLLLPRSDLAVPSELALLKAVDTWSWGERASHEEVEGLVEKIRFPM
MLPEELFELQFNLSLYWSHEALFQKKTLQALEFHTVPFQLLARYKGLNLT
EDTYKPRIYTSPTWSAFVTDSSWSARKSQLVYQSRRGPLVKYSSDYFQAP
SDYRYYPYQSFQTPQHPSFLFQDKRVSWSLVYLPTIQSCWNYGFSCSSDE
LPVLGLTKSGGSDRTIAYENKALMLCEGLFVADVTDFEGWKAAIPSALDT
NSSKSTSSFPCPAGHFNGFRTVIRPFYLTNSSGVDGGGGHHHHHHC*
>C*His-G3BP
(SEQ ID NO: 10)
MTPPRLFWVWLLVAGTQGCHHHHHHGGGGVNDGDMRLADGGATNQGRVEI
FYRGQWGTVCDNLWDLTDASVVCRALGFENATQALGRAAFGQGSGPIMLD
EVQCTGTEASLADCKSLGWLKSNCRHERDAGVVCTNETRSTHTLDLSREL
SEALGQIFDSQRGCDLSISVNVQGEDALGFCGHTVILTANLEAQALWKEP
GSNVTMSVDAECVPMVRDLLRYFYSRRIDITLSSVKCFHKLASAYGARQL
QGYCASLFAILLPQDPSFQMPLDLYAYAVATGDALLEKLCLQFLAWNFEA
LTQAEAWPSVPTDLLQLLLPRSDLAVPSELALLKAVDTWSWGERASHEEV
EGLVEKIRFPMMLPEELFELQFNLSLYWSHEALFQKKTLQALEFHTVPFQ
LLARYKGLNLTEDTYKPRIYTSPTWSAFVTDSSWSARKSQLVYQSRRGPL
VKYSSDYFQAPSDYRYYPYQSFQTPQHPSFLFQDKRVSWSLVYLPTIQSC
WNYGFSCSSDELPVLGLTKSGGSDRTIAYENKALMLCEGLFVADVTDFEG
WKAAIPSALDTNSSKSTSSFPCPAGHFNGFRTVIRPFYLTNSSGVDGGGG
HHHHHH*

5. Recombinant G3BP with PADRE and free terminal Cys

>C*His PADRE-G3BP
(SEQ ID NO: 11)
MTPPRLFWVWLLVAGTQGCHHHHHHGGSGAKFVAAWTLKAAAGSGVNDGD
MRLADGGATNQGRVEIFYRGQWGTVCDNLWDLTDASVVCRALGFENATQA
LGRAAFGQGSGPIMLDEVQCTGTEASLADCKSLGWLKSNCRHERDAGVVC
TNETRSTHTLDLSRELSEALGQIFDSQRGCDLSISVNVQGEDALGFCGHT
VILTANLEAQALWKEPGSNVTMSVDAECVPMVRDLLRYFYSRRIDITLSS
VKCFHKLASAYGARQLQGYCASLFAILLPQDPSFQMPLDLYAYAVATGDA
LLEKLCLQFLAWNFEALTQAEAWPSVPTDLLQLLLPRSDLAVPSELALLK
AVDTWSWGERASHEEVEGLVEKIRFPMMLPEELFELQFNLSLYWSHEALF
QKKTLQALEFHTVPFQLLARYKGLNLTEDTYKPRIYTSPTWSAFVTDSSW
SARKSQLVYQSRRGPLVKYSSDYFQAPSDYRYYPYQSFQTPQHPSFLFQD
KRVSWSLVYLPTIQSCWNYGFSCSSDELPVLGLTKSGGSDRTIAYENKAL
MLCEGLFVADVTDFEGWKAAIPSALDTNSSKSTSSFPCPAGHFNGFRTVI
RPFYLTNSSGVD*
>PADRE-G3BP His*C
(SEQ ID NO: 12)
MTPPRLFWVWLLVAGTQGAKFVAAWTLKAAAGSGVNDGDMRLADGGATNQ
GRVEIFYRGQWGTVCDNLWDLTDASVVCRALGFENATQALGRAAFGQGSG
PIMLDEVQCTGTEASLADCKSLGWLKSNCRHERDAGVVCTNETRSTHTLD
LSRELSEALGQIFDSQRGCDLSISVNVQGEDALGFCGHTVILTANLEAQA
LWKEPGSNVTMSVDAECVPMVRDLLRYFYSRRIDITLSSVKCFHKLASAY
GARQLQGYCASLFAILLPQDPSFQMPLDLYAYAVATGDALLEKLCLQFLA
WNFEALTQAEAWPSVPTDLLQLLLPRSDLAVPSELALLKAVDTWSWGERA
SHEEVEGLVEKIRFPMMLPEELFELQFNLSLYWSHEALFQKKTLQALEFH
TVPFQLLARYKGLNLTEDTYKPRIYTSPTWSAFVTDSSWSARKSQLVYQS
RRGPLVKYSSDYFQAPSDYRYYPYQSFQTPQHPSFLFQDKRVSWSLVYLP
TIQSCWNYGFSCSSDELPVLGLTKSGGSDRTIAYENKALMLCEGLFVADV
TDFEGWKAAIPSALDTNSSKSTSSFPCPAGHFNGFRTVIRPFYLTNSSGV
DGGGGHHHHHHC*
>C*PADRE-G3BP His
(SEQ ID NO: 13)
MTPPRLFWVWLLVAGTQGCAKFVAAWTLKAAAGSGVNDGDMRLADGGATN
QGRVEIFYRGQWGTVCDNLWDLTDASVVCRALGFENATQALGRAAFGQGS
GPIMLDEVQCTGTEASLADCKSLGWLKSNCRHERDAGVVCTNETRSTHTL
DLSRELSEALGQIFDSQRGCDLSISVNVQGEDALGFCGHTVILTANLEAQ
ALWKEPGSNVTMSVDAECVPMVRDLLRYFYSRRIDITLSSVKCFHKLASA
YGARQLQGYCASLFAILLPQDPSFQMPLDLYAYAVATGDALLEKLCLQFL
AWNFEALTQAEAWPSVPTDLLQLLLPRSDLAVPSELALLKAVDTWSWGER
ASHEEVEGLVEKIRFPMMLPEELFELQFNLSLYWSHEALFQKKTLQALEF
HTVPFQLLARYKGLNLTEDTYKPRIYTSPTWSAFVTDSSWSARKSQLVYQ
SRRGPLVKYSSDYFQAPSDYRYYPYQSFQTPQHPSFLFQDKRVSWSLVYL
PTIQSCWNYGFSCSSDELPVLGLTKSGGSDRTIAYENKALMLCEGLFVAD
VTDFEGWKAAIPSALDTNSSKSTSSFPCPAGHFNGFRTVIRPFYLTNSSG
VDGGGGHHHHHH*
>G3BP-Padre His*C
(SEQ ID NO: 14)
MTPPRLFWVWLLVAGTQGVNDGDMRLADGGATNQGRVEIFYRGQWGTVCD
NLWDLTDASVVCRALGFENATQALGRAAFGQGSGPIMLDEVQCTGTEASL
ADCKSLGWLKSNCRHERDAGVVCTNETRSTHTLDLSRELSEALGQIFDSQ
RGCDLSISVNVQGEDALGFCGHTVILTANLEAQALWKEPGSNVTMSVDAE
CVPMVRDLLRYFYSRRIDITLSSVKCFHKLASAYGARQLQGYCASLFAIL
LPQDPSFQMPLDLYAYAVATGDALLEKLCLQFLAWNFEALTQAEAWPSVP
TDLLQLLLPRSDLAVPSELALLKAVDTWSWGERASHEEVEGLVEKIRFPM
MLPEELFELQFNLSLYWSHEALFQKKTLQALEFHTVPFQLLARYKGLNLT
EDTYKPRIYTSPTWSAFVTDSSWSARKSQLVYQSRRGPLVKYSSDYFQAP
SDYRYYPYQSFQTPQHPSFLFQDKRVSWSLVYLPTIQSCWNYGFSCSSDE
LPVLGLTKSGGSDRTIAYENKALMLCEGLFVADVTDFEGWKAAIPSALDT
NSSKSTSSFPCPAGHFNGFRTVIRPFYLTNSSGVDGSGAKFVAAWTLKAA
AGGGGHHHHHHC*

6. Recombinant G3BP with TT (tetanus toxoid) peptide P30 and free terminal Cys

>TT-P30 G3BP His*C
(SEQ ID NO: 15)
MTPPRLFWVWLLVAGTQGFNNFTVSFWLRVPKVSASHLEGSGVNDGDMRL
ADGGATNQGRVEIFYRGQWGTVCDNLWDLTDASVVCRALGFENATQALGR
AAFGQGSGPIMLDEVQCTGTEASLADCKSLGWLKSNCRHERDAGVVCTNE
TRSTHTLDLSRELSEALGQIFDSQRGCDLSISVNVQGEDALGFCGHTVIL
TANLEAQALWKEPGSNVTMSVDAECVPMVRDLLRYFYSRRIDITLSSVKC
FHKLASAYGARQLQGYCASLFAILLPQDPSFQMPLDLYAYAVATGDALLE
KLCLQFLAWNFEALTQAEAWPSVPTDLLQLLLPRSDLAVPSELALLKAVD
TWSWGERASHEEVEGLVEKIRFPMMLPEELFELQFNLSLYWSHEALFQKK
TLQALEFHTVPFQLLARYKGLNLTEDTYKPRIYTSPTWSAFVTDSSWSAR
KSQLVYQSRRGPLVKYSSDYFQAPSDYRYYPYQSFQTPQHPSFLFQDKRV
SWSLVYLPTIQSCWNYGFSCSSDELPVLGLTKSGGSDRTIAYENKALMLC
EGLFVADVTDFEGWKAAIPSALDTNSSKSTSSFPCPAGHFNGFRTVIRPF
YLTNSSGVDGGGGHHHHHHC*
>C*TT-P30 G3BP His
(SEQ ID NO: 16)
MTPPRLFWVWLLVAGTQGCFNNFTVSFWLRVPKVSASHLEGSGVNDGDMR
LADGGATNQGRVEIFYRGQWGTVCDNLWDLTDASVVCRALGFENATQALG
RAAFGQGSGPIMLDEVQCTGTEASLADCKSLGWLKSNCRHERDAGVVCTN
ETRSTHTLDLSRELSEALGQIFDSQRGCDLSISVNVQGEDALGFCGHTVI
LTANLEAQALWKEPGSNVTMSVDAECVPMVRDLLRYFYSRRIDITLSSVK
CFHKLASAYGARQLQGYCASLFAILLPQDPSFQMPLDLYAYAVATGDALL
EKLCLQFLAWNFEALTQAEAWPSVPTDLLQLLLPRSDLAVPSELALLKAV
DTWSWGERASHEEVEGLVEKIRFPMMLPEELFELQFNLSLYWSHEALFQK
KTLQALEFHTVPFQLLARYKGLNLTEDTYKPRIYTSPTWSAFVTDSSWSA
RKSQLVYQSRRGPLVKYSSDYFQAPSDYRYYPYQSFQTPQHPSFLFQDKR
VSWSLVYLPTIQSCWNYGFSCSSDELPVLGLTKSGGSDRTIAYENKALML
CEGLFVADVTDFEGWKAAIPSALDTNSSKSTSSFPCPAGHFNGFRTVIRP
FYLTNSSGVDGGGGHHHHHH*
>C*His TT-P30 G3BP
(SEQ ID NO: 17)
MTPPRLFWVWLLVAGTQGCHHHHHHGGSGFNNFTVSFWLRVPKVSASHLE
GSGVNDGDMRLADGGATNQGRVEIFYRGQWGTVCDNLWDLTDASVVCRAL
GFENATQALGRAAFGQGSGPIMLDEVQCTGTEASLADCKSLGWLKSNCRH
ERDAGVVCTNETRSTHTLDLSRELSEALGQIFDSQRGCDLSISVNVQGED
ALGFCGHTVILTANLEAQALWKEPGSNVTMSVDAECVPMVRDLLRYFYSR
RIDITLSSVKCFHKLASAYGARQLQGYCASLFAILLPQDPSFQMPLDLYA
YAVATGDALLEKLCLQFLAWNFEALTQAEAWPSVPTDLLQLLLPRSDLAV
PSELALLKAVDTWSWGERASHEEVEGLVEKIRFPMMLPEELFELQFNLSL
YWSHEALFQKKTLQALEFHTVPFQLLARYKGLNLTEDTYKPRIYTSPTWS
AFVTDSSWSARKSQLVYQSRRGPLVKYSSDYFQAPSDYRYYPYQSFQTPQ
HPSFLFQDKRVSWSLVYLPTIQSCWNYGFSCSSDELPVLGLTKSGGSDRT
IAYENKALMLCEGLFVADVTDFEGWKAAIPSALDTNSSKSTSSFPCPAGH
FNGFRTVIRPFYLTNSSGVD*
>G3BP TT-P30 His*C
(SEQ ID NO: 18)
MTPPRLFWVWLLVAGTQGVNDGDMRLADGGATNQGRVEIFYRGQWGTVCD
NLWDLTDASVVCRALGFENATQALGRAAFGQGSGPIMLDEVQCTGTEASL
ADCKSLGWLKSNCRHERDAGVVCTNETRSTHTLDLSRELSEALGQIFDSQ
RGCDLSISVNVQGEDALGFCGHTVILTANLEAQALWKEPGSNVTMSVDAE
CVPMVRDLLRYFYSRRIDITLSSVKCFHKLASAYGARQLQGYCASLFAIL
LPQDPSFQMPLDLYAYAVATGDALLEKLCLQFLAWNFEALTQAEAWPSVP
TDLLQLLLPRSDLAVPSELALLKAVDTWSWGERASHEEVEGLVEKIRFPM
MLPEELFELQFNLSLYWSHEALFQKKTLQALEFHTVPFQLLARYKGLNLT
EDTYKPRIYTSPTWSAFVTDSSWSARKSQLVYQSRRGPLVKYSSDYFQAP
SDYRYYPYQSFQTPQHPSFLFQDKRVSWSLVYLPTIQSCWNYGFSCSSDE
LPVLGLTKSGGSDRTIAYENKALMLCEGLFVADVTDFEGWKAAIPSALDT
NSSKSTSSFPCPAGHFNGFRTVIRPFYLTNSSGVDGSGENNFTVSEWLRV
PKVSASHLEGGGGHHHHHHC*

In one case, Applicants have compelling preliminary data regarding the glycan reactive and Env trimer specific bNAb, PGT151, if reactive with a glycoprotein that is up-regulated by and during HIV-1 infection. Applicants propose to break tolerance to this protein by providing heterologous help, as previously shown for ubiquitin and other common and abundant self-antigens. Applicants demonstrate proof-of-principle that self-glycoprotein cross-reactivity is a key step in the re-elicitation of these infrequent and unusual N glycan and often high mannose-reactive bnABs.

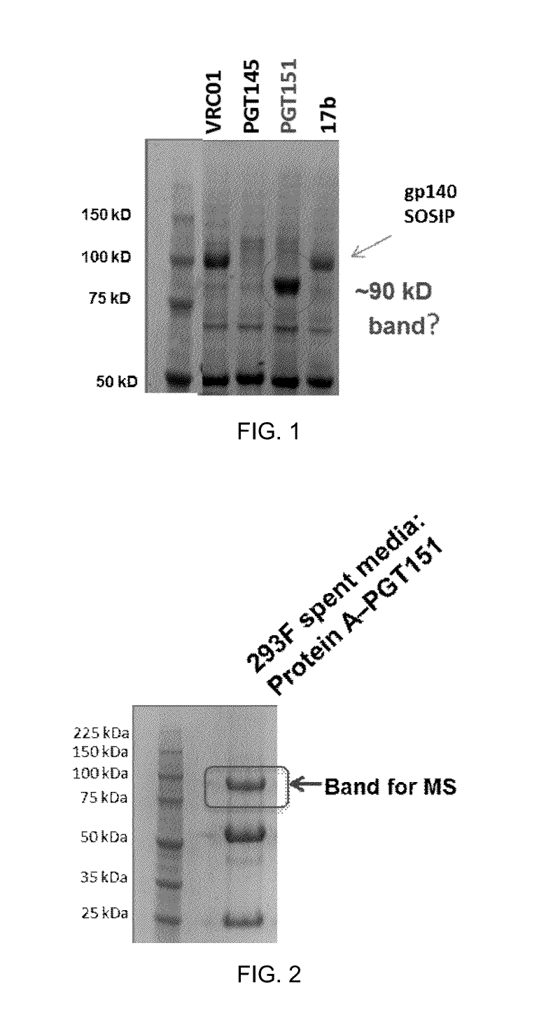

Applicants identify recognition of a non-HIV protein by one of the new bNABs in mammalian cell culture supernatants or tissues initially with a screen. To begin Applicants' screen for potential soluble protein or glycoprotein ligands to the PGT antibodies, Applicants began searching using the recently described, highly glycan recognizing bNAb, PGT 151. Applicants began with PGT 151 since it appeared to recognize a mostly N-glycan epitope and it possesses a long HCDR3 and is heavily somatically mutated. Accordingly, Applicants performed immunoprecipitation (IP) of “spent tissue culture (TC) supernatants” from several cell-types, including the commonly used human endothelial kidney cell line transformed with large T antigen, known as HEK 293T cells. Applicants used the so-called 293F cells since these are adapted to serum-free culture conditions and secret numerous mammalian proteins into the tissue culture media. Using media from tissue culture of these cells, either expressing HIV Env trimers or not, Applicants performed IPs with several mAbs, and Applicants observed that in particular, PGT151 co-precipitated an unaccounted for but somewhat diffuse band on reducing SDS page gels that migrated considerably slower in the Commassie blue-stained gel compared to the PGT 151 heavy chain, but more rapidly than co-expressed HIV-1 Env trimers with an apparent MW of approximately 90 kD (see FIG. 1). Applicants used the mAbs VRC01 and 17b as negative controls for the 90 kD band but as positive controls for non-trimeric Env. The mAb PGT145 was used to detect low levels of trimers in the 16055 SOSIP co-expression experiment.

Since the 90 kD band was diffuse in appearance on the SDS gel, Applicants suspected that it was a glycoprotein as Applicants were familiar with gp120 which appears similar due to its high level of N-linked glycosylation (FIG. 1). Following the observation of this apparent glycoprotein band Applicants excised the band from the gel (FIG. 2) and subjected the putative protein to mass spectrometry and N-terminal sequencing. The mass spectrometry yielded extremely interesting and provocative results by identifying the co-precipitated ligand to be galactose 3 binding protein (gal3BP) with over 50% coverage, a glycoprotein possessing a major gene isomer of 90 kD possessing 6-7 N-linked glycan sites. The natural ligand for gal3BP is the mammalian lectin, galectin 3 (gal 3). Thus one can envision that PGT151 recognizes gal3BP with lectin-like qualities, perhaps in a manner similar to its recognition of the HIV-1 heavily glycosylated trimer. Interestingly, gal3BP is up-regulated upon HIV infection and is detected at elevated levels in the serum of HIV-1 infected individuals. Both gal3 and gal3BP are involved in cell to cell adhesion as part of their normal function and are involved in metastases during the spread of some cancers. The gal3bp glycoprotein is also elevated in certain cancers and, in these cases, is known as MAC2. Note that in this scenario, Applicants have a mammalian/human self-glycoprotein that is well recognized by the HIV-1 broadly neutralizing PGT 151 which is expressed at high levels during HIV infection itself, perhaps making it an abundant protein to be recognized in a cross-reactive manner during affinity maturation of PGT151 by the HIV Env trimer. Or perhaps co-affinity maturation of PGT151 on both Env and gal3bp occurs; in fact then Env could provide T cell help so that PGT151 could affinity mature on both glycoproteins, especially since peripheral tolerance in the GC to self is often controlled by the lack of CD4 T help as these cells have become anergic or deleted from the repertoire in entirety, due to high-affinity self-reactivity during thymic education.

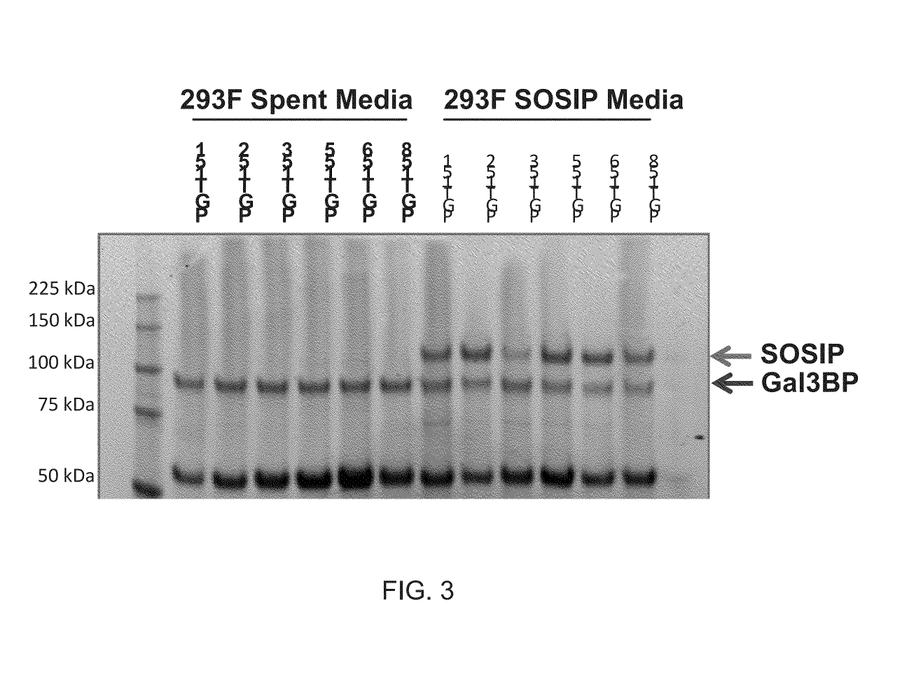

Applicants then assessed if the entire set of PGT151 family members could recognize well-formed Env trimers (JRFL SOSIP) and the 90 kD putative gal3BP glycoprotein. As shown in FIG. 3, all the PGT 151 family members (PGT 151- well recognized both the 90 kD band from spent TC supernatants (left) and co-IP'd SOSIP trimers and the 90 kD band if and when Env trimers were co-expressed by transient transfection. Note that for some PGT 151 family members, the Env was less well recognized (PGT 153) and more gal3BP was co-IP'd whereas for PGT 152, the Env was better recognized when both glycoproteins were present in the TC supernatants. These data suggested that there was co-recognition of both the Env SOSIP trimer and gal3BP and, as well, perhaps cross-competition for PGT 151 recognition (see FIG. 3). Due to this apparent high-affinity recognition of a self-protein, Applicants assessed recognition of putative PGT151 unmutated ancestral antibodies (UAs) of both gal3BP and SOSIP trimers as well, but could detect no recognition by IP (not shown). This result suggested that the cross recognition of gal3BP by PGT151 occurred during the affinity maturation in the germinal center (GC) reaction during the process of affinity maturation and the breaking of peripheral tolerance. These data suggest that there does exist B cells capable of gal3BP recognition and activation, perhaps of anergic B cells, revealing a potential means to activate or drive such B cells from the naïve repertoire.

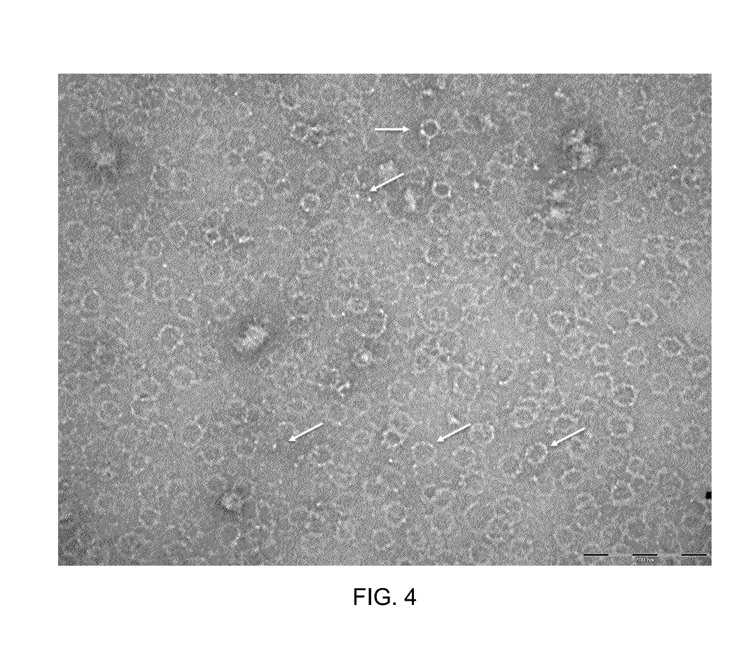

Next, Applicants performed preliminary electron microscopy of gal3BP with PGT151 Fab and observed that gal3BP forms oligomeric rings as previously reported for this glycoprotein (FIG. 4). Resolution of the PGT151 Fab relative to these rings was unclear and is further refined.

In this application, Applicants probe cell line supernatants and cell surfaces with all the existing bNAbs that co-recognize HIV-1 polypeptide surfaces and N-linked glycans as determined by selected mapping techniques such as N-glycan deletion viruses at residues 332 and 301 as previously described.

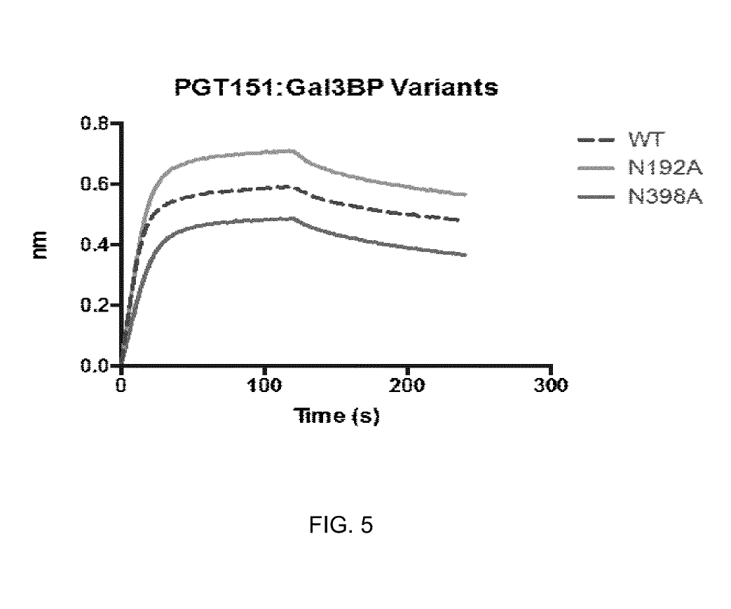

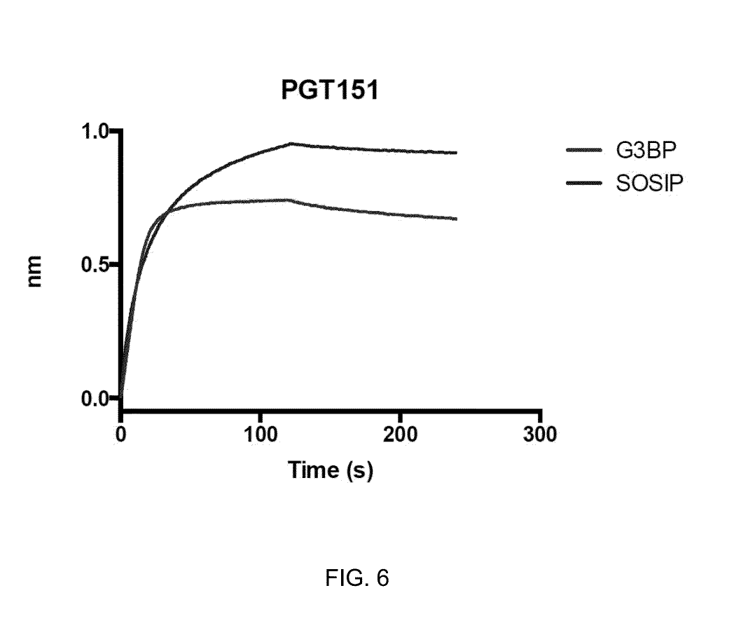

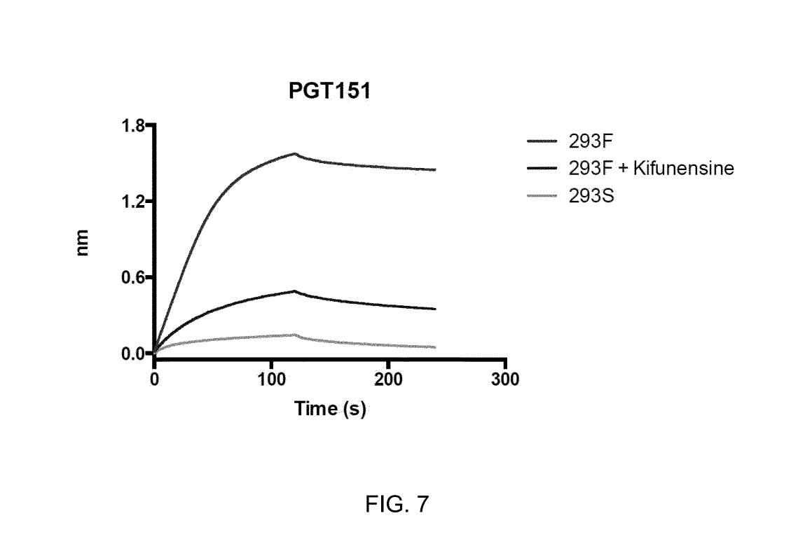

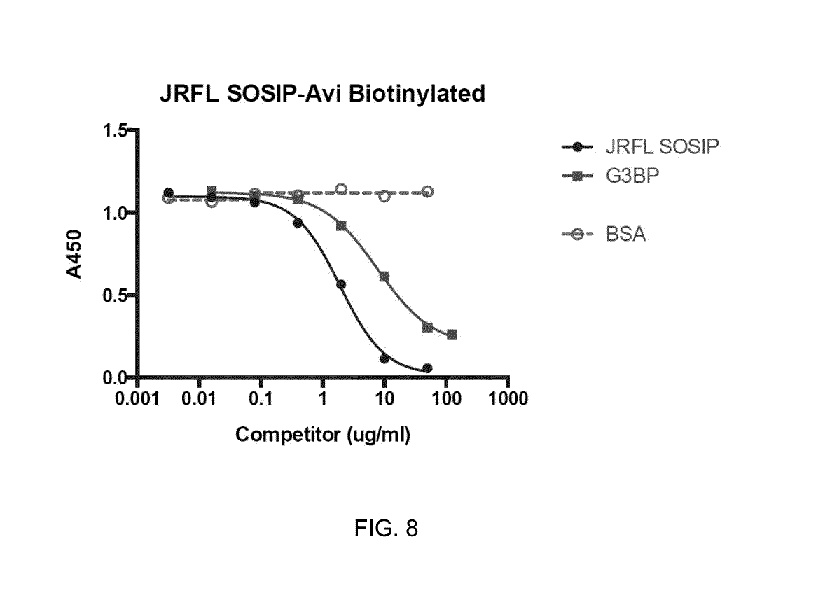

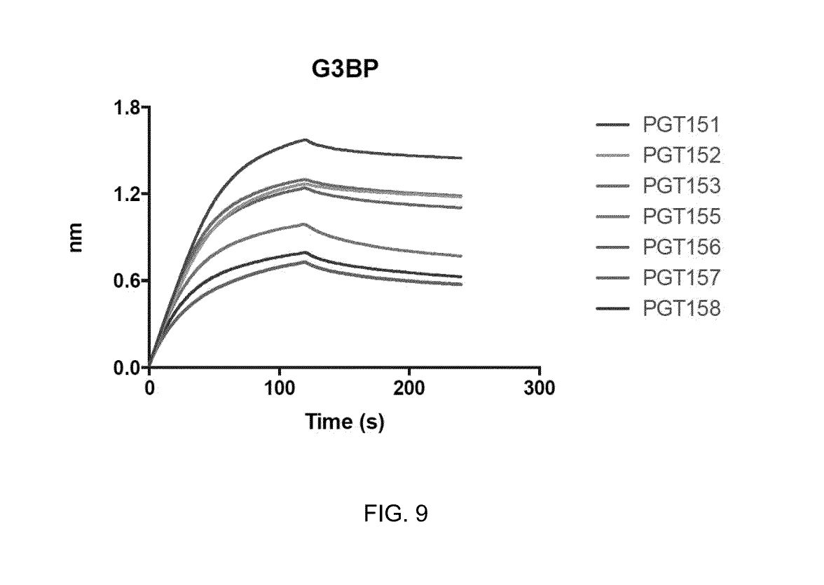

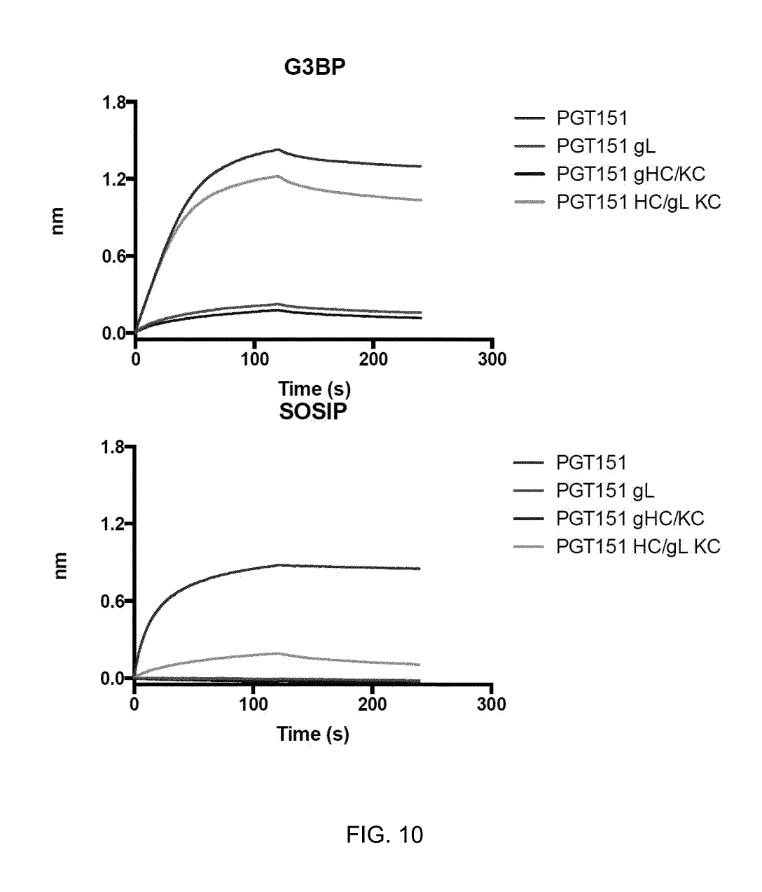

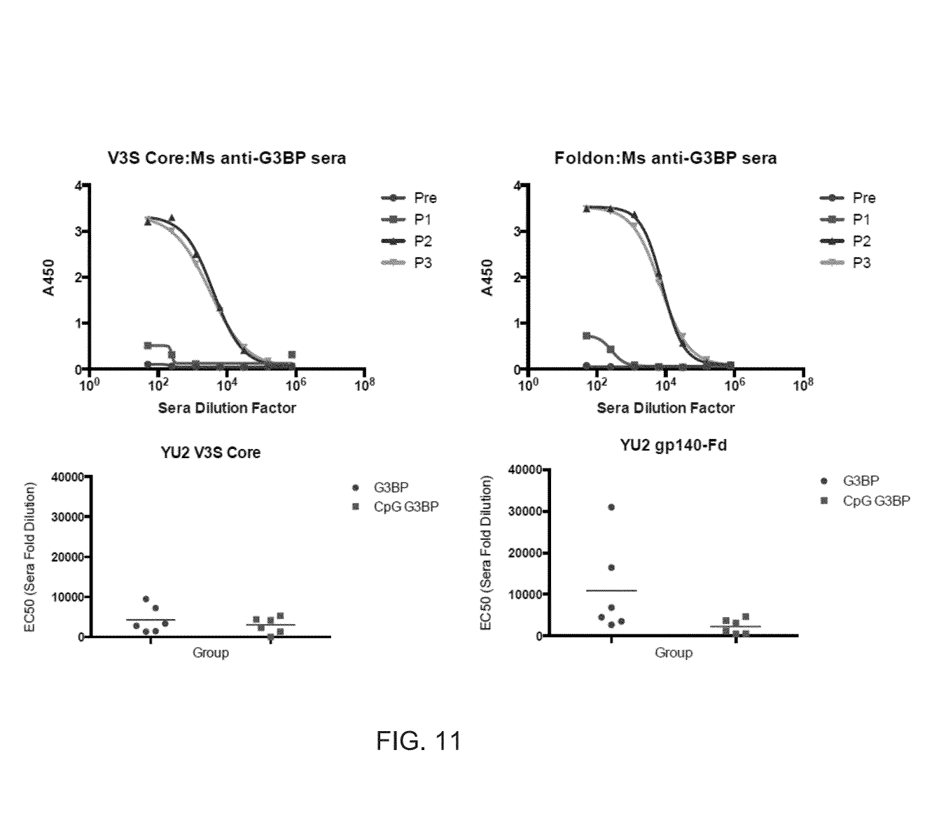

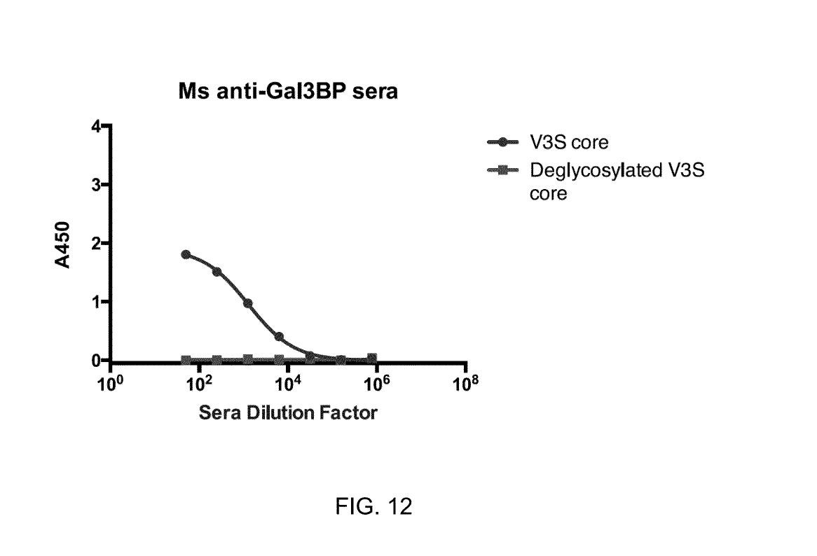

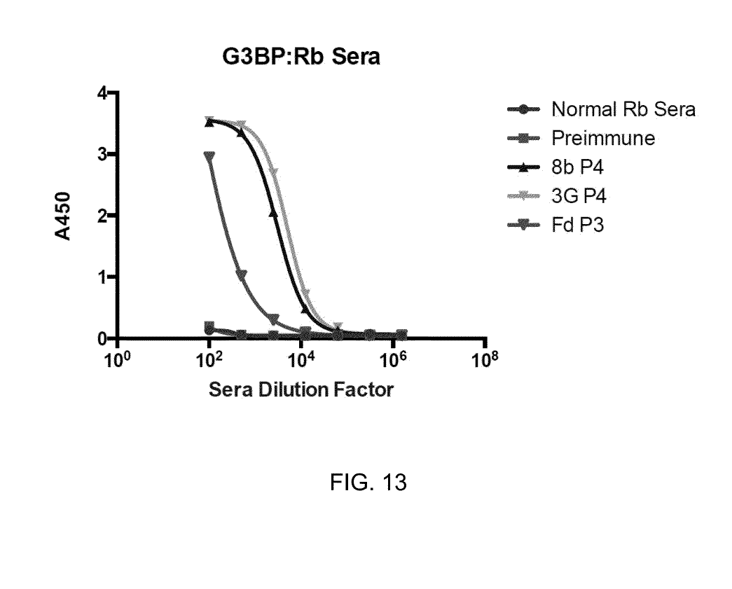

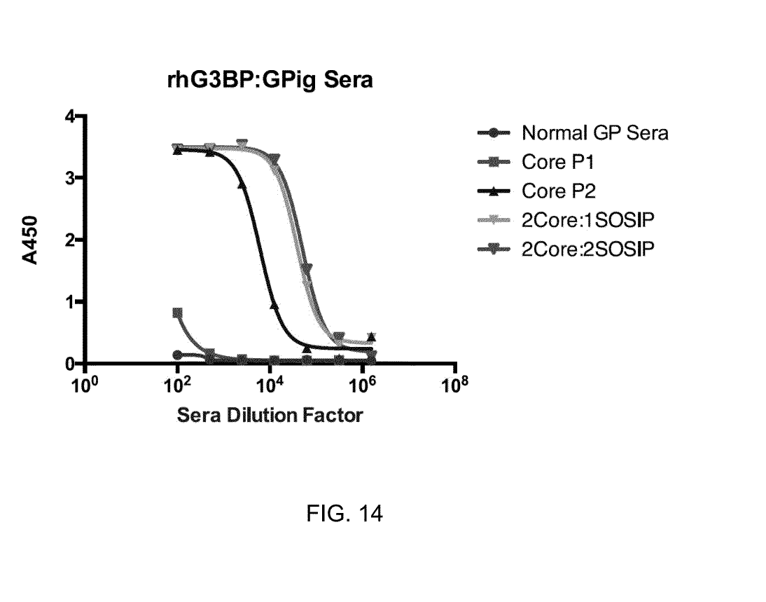

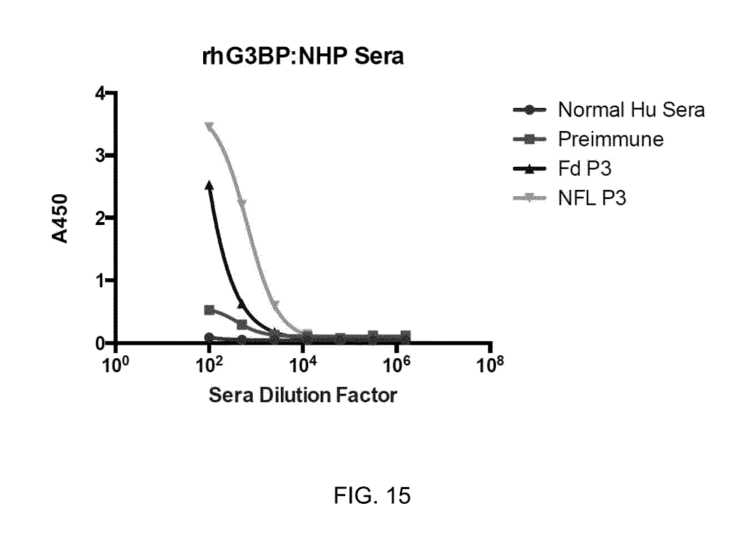

Applicants characterize and comprehensively map the bNAb:protein/glycoprotein interactive surfaces by performing site-directed mutagenesis of recombinant gal3BP and detected decreases in affinity binding by PGT151 following the deletion of three individual N-linked glycosylation sites on gal3BP (FIG. 5). These data provide the basis for future fine-mapping of the PGT 151 epitope on gal3BP.

Applicants perform site-directed mutagenesis of individual gal3BP N-linked glycans by Quikchange site-directed mutagenesis and evaluate recognition by PGT151 and family members of WT and altered gal3BP. Applicants also assess the impact of N-glycan deletion on gal3BP expression, folding and ring formation by EM.

The invention also encompasses additional lineage analysis of PFT151 family member patient PBMCs, determining interactions with gal3, does gal3 recognize HIV1-Env with any detectable affinity, protein arrays displaying over 8000 mammalian proteins and glycoproteins, tissue sections of normal and human tumor tissue sections, screening normal human serum versus serum derived from HIV infection from the PGT 151 patient and other individuals and screening both the supernatants and PHA T cell blasts from patient PGT151 and other normal and HIV-1-infected individuals.

Applicants use the bNAb-recognized self-protein, coupled with both heterologous help to break tolerance and well-ordered trimer boosting, to re-elicit bNAbs in vivo. The invention also encompasses immunogenicity, breaking tolerance using heterologous T cell helper epitopes or multivalent gal3BP array to stimulate anergic or low affinity B cells, prime boosting with Env and low affinity priming with diverse gal3BP glycoproteins from other species to better overcome B cell anergy/deletion coupled with heterologous T cell help.

If tolerance cannot be broken by priming with gal3BP containing known poly-reactive_MHC class II peptides, such as PADRE or TT or HA class II peptides are also contemplated.

In one embodiment the heterologous cell help may be from flu HA (13 residues or so, or multiple T helper epitopes) or PADRE (pan-DR epitopes) or the TT peptide by genetic fusion to the non-HIV self-protein, either at the C- or N-terminus, that will then be expressed from 293F cells as a recombinant fusion protein containing these so-called “promiscuous” T helper peptides (PADRE, TT, HA). Promiscuous T helper peptides are called this because their anchor residues bind to many MHC class II alleles and therefore can provide T cell help in outbred populations. Such heterologous T help will be necessary to generate an immune response to a human self protein in humans. Exemplary sequences for these fusion proteins are provided in the present application.

Assays for screening for neutralizing antibodies are known in the art. A neutralization assay approach has been described previously (Binley J M, et al., (2004). Comprehensive Cross-Clade Neutralization Analysis of a Panel of Anti-Human Immunodeficiency Virus Type 1 Monoclonal Antibodies. J. Virol. 78: 13232-13252). Pseudotyped viruses may be generated by co-transfecting cells with at least two plasmids encoding the soluble Env cDNA of the present invention and the rest of the HIV genome separately. In the HIV genome encoding vector, the Env gene may be replaced by the firefly luciferase gene. Transfectant supernatants containing pseudotyped virus may be co-incubated overnight with B cell supernatants derived from activation of an infected donor's primary peripheral blood mononuclear cells (PBMCs). Cells stably transfected with and expressing CD4 plus the CCR5 and CXCR4 coreceptors may be added to the mixture and incubated for 3 days at 37° C. Infected cells may be quantified by luminometry.

In yet another embodiment, the present invention also encompassed the use of the variants described herein as immunogens, advantageously as HIV-1 vaccine components.

The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antibody” includes intact molecules as well as fragments thereof, such as Fab, F(ab′)₂, Fv and scFv which are capable of binding the epitope determinant. These antibody fragments retain some ability to selectively bind with its antigen or receptor and include, for example:

Fab, the fragment which contains a monovalent antigen-binding fragment of an antibody molecule can be produced by digestion of whole antibody with the enzyme papain to yield an intact light chain and a portion of one heavy chain;

Fab′, the fragment of an antibody molecule can be obtained by treating whole antibody with pepsin, followed by reduction, to yield an intact light chain and a portion of the heavy chain; two Fab' fragments are obtained per antibody molecule;

F(ab′)₂, the fragment of the antibody that can be obtained by treating whole antibody with the enzyme pepsin without subsequent reduction; F(ab′)₂ is a dimer of two Fab′ fragments held together by two disulfide bonds;

scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference).

A “neutralizing antibody” may inhibit the entry of HIV-1 virus F with a neutralization index >1.5 or >2.0. Broad and potent neutralizing antibodies may neutralize greater than about 50% of HIV-1 viruses (from diverse clades and different strains within a clade) in a neutralization assay. The inhibitory concentration of the monoclonal antibody may be less than about 25 mg/ml to neutralize about 50% of the input virus in the neutralization assay.

It should be understood that the proteins, including the antibodies and/or antigens of the invention may differ from the exact sequences illustrated and described herein. Thus, the invention contemplates deletions, additions and substitutions to the sequences shown, so long as the sequences function in accordance with the methods of the invention. In this regard, particularly preferred substitutions are generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid can be single-stranded, or partially or completely double-stranded (duplex). Duplex nucleic acids can be homoduplex or heteroduplex.

As used herein the term “transgene” may be used to refer to “recombinant” nucleotide sequences that may be derived from any of the nucleotide sequences encoding the proteins of the present invention. The term “recombinant” means a nucleotide sequence that has been manipulated “by man” and which does not occur in nature, or is linked to another nucleotide sequence or found in a different arrangement in nature. It is understood that manipulated “by man” means manipulated by some artificial means, including by use of machines, codon optimization, restriction enzymes, etc.

For example, in one embodiment the nucleotide sequences may be mutated such that the activity of the encoded proteins in vivo is abrogated. In another embodiment the nucleotide sequences may be codon optimized, for example the codons may be optimized for human use. In preferred embodiments the nucleotide sequences of the invention are both mutated to abrogate the normal in vivo function of the encoded proteins, and codon optimized for human use. For example, each of the Gag, Pol, Env, Nef, RT, and Int sequences of the invention may be altered in these ways.

As regards codon optimization, the nucleic acid molecules of the invention have a nucleotide sequence that encodes the antigens of the invention and can be designed to employ codons that are used in the genes of the subject in which the antigen is to be produced. Many viruses, including HIV and other lentiviruses, use a large number of rare codons and, by altering these codons to correspond to codons commonly used in the desired subject, enhanced expression of the antigens can be achieved. In a preferred embodiment, the codons used are “humanized” codons, i.e., the codons are those that appear frequently in highly expressed human genes (Andre et al., J. Virol. 72:1497-1503, 1998) instead of those codons that are frequently used by HIV. Such codon usage provides for efficient expression of the transgenic HIV proteins in human cells. Any suitable method of codon optimization may be used. Such methods, and the selection of such methods, are well known to those of skill in the art. In addition, there are several companies that will optimize codons of sequences, such as Geneart (geneart.com). Thus, the nucleotide sequences of the invention can readily be codon optimized.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993;90: 5873-5877.

Another example of a mathematical algorithm used for comparison of sequences is the algorithm of Myers & Miller, CABIOS 1988;4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993;Nature Genetics 3: 266-272; Karlin & Altschul, 1993;Proc. Natl. Acad. Sci. USA 90: 5873-5877; all of which are incorporated by reference herein).

The various recombinant nucleotide sequences and antibodies and/or antigens of the invention are made using standard recombinant DNA and cloning techniques. Such techniques are well known to those of skill in the art. See for example, “Molecular Cloning: A Laboratory Manual”, second edition (Sambrook et al. 1989).

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the antibodies and/or antigens of the present invention may be used in accordance with the present invention. In certain embodiments, the antigens and/or antibodies of the present invention may be used in vitro (such as using cell-free expression systems) and/or in cultured cells grown in vitro in order to produce the encoded HIV-antigens and/or antibodies which may then be used for various applications such as in the production of proteinaceous vaccines. For such applications, any vector that allows expression of the antigens and/or antibodies in vitro and/or in cultured cells may be used.

For applications where it is desired that the antibodies and/or antigens be expressed in vivo, for example when the transgenes of the invention are used in DNA or DNA-containing vaccines, any vector that allows for the expression of the antibodies and/or antigens of the present invention and is safe for use in vivo may be used. In preferred embodiments the vectors used are safe for use in humans, mammals and/or laboratory animals.

For the antibodies and/or antigens of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” can be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the transgenes of the present invention can be under the control of a constitutive promoter or of an inducible promoter, which initiates transcription only when exposed to some particular external stimulus, such as, without limitation, antibiotics such as tetracycline, hormones such as ecdysone, or heavy metals. The promoter can also be specific to a particular cell-type, tissue or organ. Many suitable promoters and enhancers are known in the art, and any such suitable promoter or enhancer may be used for expression of the transgenes of the invention. For example, suitable promoters and/or enhancers can be selected from the Eukaryotic Promoter Database (EPDB).

The present invention relates to a recombinant vector expressing a foreign epitope. Advantageously, the epitope is an HIV epitope. In an advantageous embodiment, the HIV epitope is a soluble envelope glycoprotein, however, the present invention may encompass additional HIV antigens, epitopes or immunogens. Advantageously, the HIV epitope is an HIV antigen, HIV epitope or an HIV immunogen, such as, but not limited to, the HIV antigens, HIV epitopes or HIV immunogens of U.S. Pat. Nos. 7,341,731; 7,335,364; 7,329,807; 7,323,553; 7,320,859; 7,311,920; 7,306,798; 7,285,646; 7,285,289; 7,285,271; 7,282,364; 7,273,695; 7,270,997; 7,262,270; 7,244,819; 7,244,575; 7,232,567; 7,232,566; 7,223,844; 7,223,739; 7,223,534; 7,223,368; 7,220,554; 7,214,530; 7,211,659; 7,211,432; 7,205,159; 7,198,934; 7,195,768; 7,192,555; 7,189,826; 7,189,522; 7,186,507; 7,179,645; 7,175,843; 7,172,761; 7,169,550; 7,157,083; 7,153,509; 7,147,862; 7,141,550; 7,129,219; 7,122,188; 7,118,859; 7,118,855; 7,118,751; 7,118,742; 7,105,655; 7,101,552; 7,097,971; 7,097,842; 7,094,405; 7,091,049; 7,090,648; 7,087,377; 7,083,787; 7,070,787; 7,070,781; 7,060,273; 7,056,521; 7,056,519; 7,049,136; 7,048,929; 7,033,593; 7,030,094; 7,022,326; 7,009,037; 7,008,622; 7,001,759; 6,997,863; 6,995,008; 6,979,535; 6,974,574; 6,972,126; 6,969,609; 6,964,769; 6,964,762; 6,958,158; 6,956,059; 6,953,689; 6,951,648; 6,946,075; 6,927,031; 6,919,319; 6,919,318; 6,919,077; 6,913,752; 6,911,315; 6,908,617; 6,908,612; 6,902,743; 6,900,010; 6,893,869; 6,884,785; 6,884,435; 6,875,435; 6,867,005; 6,861,234; 6,855,539; 6,841,381 6,841,345; 6,838,477; 6,821,955; 6,818,392; 6,818,222; 6,815,217; 6,815,201; 6,812,026; 6,812,025; 6,812,024; 6,808,923; 6,806,055; 6,803,231; 6,800,613; 6,800,288; 6,797,811; 6,780,967; 6,780,598; 6,773,920; 6,764,682; 6,761,893; 6,753,015; 6,750,005; 6,737,239; 6,737,067; 6,730,304; 6,720,310; 6,716,823; 6,713,301; 6,713,070; 6,706,859; 6,699,722; 6,699,656; 6,696,291; 6,692,745; 6,670,181; 6,670,115; 6,664,406; 6,657,055; 6,657,050; 6,656,471; 6,653,066; 6,649,409; 6,649,372; 6,645,732; 6,641,816; 6,635,469; 6,613,530; 6,605,427; 6,602,709; 6,602,705; 6,600,023; 6,596,477; 6,596,172; 6,593,103; 6,593,079; 6,579,673; 6,576,758; 6,573,245; 6,573,040; 6,569,418; 6,569,340; 6,562,800; 6,558,961; 6,551,828; 6,551,824; 6,548,275; 6,544,780; 6,544,752; 6,544,728; 6,534,482; 6,534,312; 6,534,064; 6,531,572; 6,531,313; 6,525,179; 6,525,028; 6,524,582; 6,521,449; 6,518,030; 6,518,015; 6,514,691; 6,514,503; 6,511,845; 6,511,812; 6,511,801; 6,509,313; 6,506,384; 6,503,882; 6,495,676; 6,495,526; 6,495,347; 6,492,123; 6,489,131; 6,489,129; 6,482,614; 6,479,286; 6,479,284; 6,465,634; 6,461,615; 6,458,560; 6,458,527; 6,458,370; 6,451,601; 6,451,592; 6,451,323; 6,436,407; 6,432,633; 6,428,970; 6,428,952; 6,428,790; 6,420,139; 6,416,997; 6,410,318; 6,410,028; 6,410,014; 6,407,221; 6,406,710; 6,403,092; 6,399,295; 6,392,013; 6,391,657; 6,384,198; 6,380,170; 6,376,170; 6,372,426; 6,365,187; 6,358,739; 6,355,248; 6,355,247; 6,348,450; 6,342,372; 6,342,228; 6,338,952; 6,337,179; 6,335,183; 6,335,017; 6,331,404; 6,329,202; 6,329,173; 6,328,976; 6,322,964; 6,319,666; 6,319,665; 6,319,500; 6,319,494; 6,316,205; 6,316,003; 6,309,633; 6,306,625; 6,296,807; 6,294,322; 6,291,239; 6,291,157; 6,287,568; 6,284,456; 6,284,194; 6,274,337; 6,270,956; 6,270,769; 6,268,484; 6,265,562; 6,265,149; 6,262,029; 6,261,762; 6,261,571; 6,261,569; 6,258,599; 6,258,358; 6,248,332; 6,245,331; 6,242,461; 6,241,986; 6,235,526; 6,235,466; 6,232,120; 6,228,361; 6,221,579; 6,214,862; 6,214,804; 6,210,963; 6,210,873; 6,207,185; 6,203,974; 6,197,755; 6,197,531; 6,197,496; 6,194,142; 6,190,871; 6,190,666; 6,168,923; 6,156,302; 6,153,408; 6,153,393; 6,153,392; 6,153,378; 6,153,377; 6,146,635; 6,146,614; 6,143,876 6,140,059; 6,140,043; 6,139,746; 6,132,992; 6,124,306; 6,124,132; 6,121,006; 6,120,990; 6,114,507; 6,114,143; 6,110,466; 6,107,020; 6,103,521; 6,100,234; 6,099,848; 6,099,847; 6,096,291; 6,093,405; 6,090,392; 6,087,476; 6,083,903; 6,080,846; 6,080,725; 6,074,650; 6,074,646; 6,070,126; 6,063,905; 6,063,564; 6,060,256; 6,060,064; 6,048,530; 6,045,788; 6,043,347; 6,043,248; 6,042,831; 6,037,165; 6,033,672; 6,030,772; 6,030,770; 6,030,618; 6,025,141; 6,025,125; 6,020,468; 6,019,979; 6,017,543; 6,017,537; 6,015,694; 6,015,661; 6,013,484; 6,013,432; 6,007,838; 6,004,811; 6,004,807; 6,004,763; 5,998,132; 5,993,819; 5,989,806; 5,985,926; 5,985,641; 5,985,545; 5,981,537; 5,981,505; 5,981,170; 5,976,551; 5,972,339; 5,965,371; 5,962,428; 5,962,318; 5,961,979; 5,961,970; 5,958,765; 5,958,422; 5,955,647; 5,955,342; 5,951,986; 5,951,975; 5,942,237; 5,939,277; 5,939,074; 5,935,580; 5,928,930; 5,928,913; 5,928,644; 5,928,642; 5,925,513; 5,922,550; 5,922,325; 5,919,458; 5,916,806; 5,916,563; 5,914,395; 5,914,109; 5,912,338; 5,912,176; 5,912,170; 5,906,936; 5,895,650; 5,891,623; 5,888,726; 5,885,580 5,885,578; 5,879,685; 5,876,731; 5,876,716; 5,874,226; 5,872,012; 5,871,747; 5,869,058; 5,866,694; 5,866,341; 5,866,320; 5,866,319; 5,866,137; 5,861,290; 5,858,740; 5,858,647; 5,858,646; 5,858,369; 5,858,368; 5,858,366; 5,856,185; 5,854,400; 5,853,736; 5,853,725; 5,853,724; 5,852,186; 5,851,829; 5,851,529; 5,849,475; 5,849,288; 5,843,728; 5,843,723; 5,843,640; 5,843,635; 5,840,480; 5,837,510; 5,837,250; 5,837,242; 5,834,599; 5,834,441; 5,834,429; 5,834,256; 5,830,876; 5,830,641; 5,830,475; 5,830,458; 5,830,457; 5,827,749; 5,827,723; 5,824,497; 5,824,304; 5,821,047; 5,817,767; 5,817,754; 5,817,637; 5,817,470; 5,817,318; 5,814,482; 5,807,707; 5,804,604; 5,804,371; 5,800,822; 5,795,955; 5,795,743; 5,795,572; 5,789,388; 5,780,279; 5,780,038; 5,776,703; 5,773,260; 5,770,572; 5,766,844; 5,766,842; 5,766,625; 5,763,574; 5,763,190; 5,762,965; 5,759,769; 5,756,666; 5,753,258; 5,750,373; 5,747,641; 5,747,526; 5,747,028; 5,736,320; 5,736,146; 5,733,760; 5,731,189; 5,728,385; 5,721,095; 5,716,826; 5,716,637; 5,716,613; 5,714,374; 5,709,879; 5,709,860; 5,709,843; 5,705,331; 5,703,057; 5,702,707 5,698,178; 5,688,914; 5,686,078; 5,681,831; 5,679,784; 5,674,984; 5,672,472; 5,667,964; 5,667,783; 5,665,536; 5,665,355; 5,660,990; 5,658,745; 5,658,569; 5,643,756; 5,641,624; 5,639,854; 5,639,598; 5,637,677; 5,637,455; 5,633,234; 5,629,153; 5,627,025; 5,622,705; 5,614,413; 5,610,035; 5,607,831; 5,606,026; 5,601,819; 5,597,688; 5,593,972; 5,591,829; 5,591,823; 5,589,466; 5,587,285; 5,585,254; 5,585,250; 5,580,773; 5,580,739; 5,580,563; 5,573,916; 5,571,667; 5,569,468; 5,558,865; 5,556,745; 5,550,052; 5,543,328; 5,541,100; 5,541,057; 5,534,406; 5,529,765; 5,523,232; 5,516,895; 5,514,541; 5,510,264; 5,500,161; 5,480,967; 5,480,966; 5,470,701; 5,468,606; 5,462,852; 5,459,127; 5,449,601; 5,447,838; 5,447,837; 5,439,809; 5,439,792; 5,418,136; 5,399,501; 5,397,695; 5,391,479; 5,384,240; 5,374,519; 5,374,518; 5,374,516; 5,364,933; 5,359,046; 5,356,772; 5,354,654; 5,344,755; 5,335,673; 5,332,567; 5,320,940; 5,317,009; 5,312,902; 5,304,466; 5,296,347; 5,286,852; 5,268,265; 5,264,356; 5,264,342; 5,260,308; 5,256,767; 5,256,561; 5,252,556; 5,230,998; 5,230,887; 5,227,159; 5,225,347; 5,221,610 5,217,861; 5,208,321; 5,206,136; 5,198,346; 5,185,147; 5,178,865; 5,173,400; 5,173,399; 5,166,050; 5,156,951; 5,135,864; 5,122,446; 5,120,662; 5,103,836; 5,100,777; 5,100,662; 5,093,230; 5,077,284; 5,070,010; 5,068,174; 5,066,782; 5,055,391; 5,043,262; 5,039,604; 5,039,522; 5,030,718; 5,030,555; 5,030,449; 5,019,387; 5,013,556; 5,008,183; 5,004,697; 4,997,772; 4,983,529; 4,983,387; 4,965,069; 4,945,082; 4,921,787; 4,918,166; 4,900,548; 4,888,290; 4,886,742; 4,885,235; 4,870,003; 4,869,903; 4,861,707; 4,853,326; 4,839,288; 4,833,072 and 4,795,739.

In another embodiment, HIV, or immunogenic fragments thereof, may be utilized as the HIV epitope. For example, the HIV nucleotides of U.S. Pat. Nos. 7,393,949, 7,374,877, 7,306,901, 7,303,754, 7,173,014, 7,122,180, 7,078,516, 7,022,814, 6,974,866, 6,958,211, 6,949,337, 6,946,254, 6,896,900, 6,887,977, 6,870,045, 6,803,187, 6,794,129, 6,773,915, 6,768,004, 6,706,268, 6,696,291, 6,692,955, 6,656,706, 6,649,409, 6,627,442, 6,610,476, 6,602,705, 6,582,920, 6,557,296, 6,531,587, 6,531,137, 6,500,623, 6,448,078, 6,429,306, 6,420,545, 6,410,013, 6,407,077, 6,395,891, 6,355,789, 6,335,158, 6,323,185, 6,316,183, 6,303,293, 6,300,056, 6,277,561, 6,270,975, 6,261,564, 6,225,045, 6,222,024, 6,194,391, 6,194,142, 6,162,631, 6,114,167, 6,114,109, 6,090,392, 6,060,587, 6,057,102, 6,054,565, 6,043,081, 6,037,165, 6,034,233, 6,033,902, 6,030,769, 6,020,123, 6,015,661, 6,010,895, 6,001,555, 5,985,661, 5,980,900, 5,972,596, 5,939,538, 5,912,338, 5,869,339, 5,866,701, 5,866,694, 5,866,320, 5,866,137, 5,864,027, 5,861,242, 5,858,785, 5,858,651, 5,849,475, 5,843,638, 5,840,480, 5,821,046, 5,801,056, 5,786,177, 5,786,145, 5,773,247, 5,770,703, 5,756,674, 5,741,706, 5,705,612, 5,693,752, 5,688,637, 5,688,511, 5,684,147, 5,665,577, 5,585,263, 5,578,715, 5,571,712, 5,567,603, 5,554,528, 5,545,726, 5,527,895, 5,527,894, 5,223,423, 5,204,259, 5,144,019, 5,051,496 and 4,942,122 are useful for the present invention.

Any epitope recognized by an HIV antibody may be used in the present invention. For example, the anti-HIV antibodies of U.S. Pat. Nos. 6,949,337, 6,900,010, 6,821,744, 6,768,004, 6,613,743, 6,534,312, 6,511,830, 6,489,131, 6,242,197, 6,114,143, 6,074,646, 6,063,564, 6,060,254, 5,919,457, 5,916,806, 5,871,732, 5,824,304, 5,773,247, 5,736,320, 5,637,455, 5,587,285, 5,514,541, 5,317,009, 4,983,529, 4,886,742, 4,870,003 and 4,795,739 are useful for the present invention. Furthermore, monoclonal anti-HIV antibodies of U.S. Pat. Nos. 7,074,556, 7,074,554, 7,070,787, 7,060,273, 7,045,130, 7,033,593, RE39,057, 7,008,622, 6,984,721, 6,972,126, 6,949,337, 6,946,465, 6,919,077, 6,916,475, 6,911,315, 6,905,680, 6,900,010, 6,825,217, 6,824,975, 6,818,392, 6,815,201, 6,812,026, 6,812,024, 6,797,811, 6,768,004, 6,703,019, 6,689,118, 6,657,050, 6,608,179, 6,600,023, 6,596,497, 6,589,748, 6,569,143, 6,548,275, 6,525,179, 6,524,582, 6,506,384, 6,498,006, 6,489,131, 6,465,173, 6,461,612, 6,458,933, 6,432,633, 6,410,318, 6,406,701, 6,395,275, 6,391,657, 6,391,635, 6,384,198, 6,376,170, 6,372,217, 6,344,545, 6,337,181, 6,329,202, 6,319,665, 6,319,500, 6,316,003, 6,312,931, 6,309,880, 6,296,807, 6,291,239, 6,261,558, 6,248,514, 6,245,331, 6,242,197, 6,241,986, 6,228,361, 6,221,580, 6,190,871, 6,177,253, 6,146,635, 6,146,627, 6,146,614, 6,143,876, 6,132,992, 6,124,132, RE36,866, 6,114,143, 6,103,238, 6,060,254, 6,039,684, 6,030,772, 6,020,468, 6,013,484, 6,008,044, 5,998,132, 5,994,515, 5,993,812, 5,985,545, 5,981,278, 5,958,765, 5,939,277, 5,928,930, 5,922,325, 5,919,457, 5,916,806, 5,914,109, 5,911,989, 5,906,936, 5,889,158, 5,876,716, 5,874,226, 5,872,012, 5,871,732, 5,866,694, 5,854,400, 5,849,583, 5,849,288, 5,840,480, 5,840,305, 5,834,599, 5,831,034, 5,827,723, 5,821,047, 5,817,767, 5,817,458, 5,804,440, 5,795,572, 5,783,670, 5,776,703, 5,773,225, 5,766,944, 5,753,503, 5,750,373, 5,747,641, 5,736,341, 5,731,189, 5,707,814, 5,702,707, 5,698,178, 5,695,927, 5,665,536, 5,658,745, 5,652,138, 5,645,836, 5,635,345, 5,618,922, 5,610,035, 5,607,847, 5,604,092, 5,601,819, 5,597,896, 5,597,688, 5,591,829, 5,558,865, 5,514,541, 5,510,264, 5,478,753, 5,374,518, 5,374,516, 5,344,755, 5,332,567, 5,300,433, 5,296,347, 5,286,852, 5,264,221, 5,260,308, 5,256,561, 5,254,457, 5,230,998, 5,227,159, 5,223,408, 5,217,895, 5,180,660, 5,173,399, 5,169,752, 5,166,050, 5,156,951, 5,140,105, 5,135,864, 5,120,640, 5,108,904, 5,104,790, 5,049,389, 5,030,718, 5,030,555, 5,004,697, 4,983,529, 4,888,290, 4,886,742 and 4,853,326, are also useful for the present invention.

The vectors used in accordance with the present invention should typically be chosen such that they contain a suitable gene regulatory region, such as a promoter or enhancer, such that the antigens and/or antibodies of the invention can be expressed.

For example, when the aim is to express the antibodies and/or antigens of the invention in vitro, or in cultured cells, or in any prokaryotic or eukaryotic system for the purpose of producing the protein(s) encoded by that antibody and/or antigen, then any suitable vector can be used depending on the application. For example, plasmids, viral vectors, bacterial vectors, protozoal vectors, insect vectors, baculovirus expression vectors, yeast vectors, mammalian cell vectors, and the like, can be used. Suitable vectors can be selected by the skilled artisan taking into consideration the characteristics of the vector and the requirements for expressing the antibodies and/or antigens under the identified circumstances.

When the aim is to express the antibodies and/or antigens of the invention in vivo in a subject, for example in order to generate an immune response against an HIV-1 antigen and/or protective immunity against HIV-1, expression vectors that are suitable for expression on that subject, and that are safe for use in vivo, should be chosen. For example, in some embodiments it may be desired to express the antibodies and/or antigens of the invention in a laboratory animal, such as for pre-clinical testing of the HIV-1 immunogenic compositions and vaccines of the invention. In other embodiments, it will be desirable to express the antibodies and/or antigens of the invention in human subjects, such as in clinical trials and for actual clinical use of the immunogenic compositions and vaccine of the invention. Any vectors that are suitable for such uses can be employed, and it is well within the capabilities of the skilled artisan to select a suitable vector. In some embodiments it may be preferred that the vectors used for these in vivo applications are attenuated to vector from amplifying in the subject. For example, if plasmid vectors are used, preferably they will lack an origin of replication that functions in the subject so as to enhance safety for in vivo use in the subject.. If viral vectors are used, preferably they are attenuated or replication-defective in the subject, again, so as to enhance safety for in vivo use in the subject.

In preferred embodiments of the present invention viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Such viruses, when used as expression vectors are innately non-pathogenic in the selected subjects such as humans or have been modified to render them non-pathogenic in the selected subjects. For example, replication-defective adenoviruses and alphaviruses are well known and can be used as gene delivery vectors.

The nucleotide sequences and vectors of the invention can be delivered to cells, for example if aim is to express and the HIV-1 antigens in cells in order to produce and isolate the expressed proteins, such as from cells grown in culture. For expressing the antibodies and/or antigens in cells any suitable transfection, transformation, or gene delivery methods can be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the antibodies and/or antigens can be carried out in any suitable type of host cells, such as bacterial cells, yeast, insect cells, and mammalian cells. The antibodies and/or antigens of the invention can also be expressed using including in vitro transcription/translation systems. All of such methods are well known by those skilled in the art, and one of skill in the art would readily be able to select a suitable method depending on the nature of the nucleotide sequences, vectors, and cell types used.

In preferred embodiments, the nucleotide sequences, antibodies and/or antigens of the invention are administered in vivo, for example where the aim is to produce an immunogenic response in a subject. A “subject” in the context of the present invention may be any animal. For example, in some embodiments it may be desired to express the transgenes of the invention in a laboratory animal, such as for pre-clinical testing of the HIV-1 immunogenic compositions and vaccines of the invention. In other embodiments, it will be desirable to express the antibodies and/or antigens of the invention in human subjects, such as in clinical trials and for actual clinical use of the immunogenic compositions and vaccine of the invention. In preferred embodiments the subject is a human, for example a human that is infected with, or is at risk of infection with, HIV-1.

For such in vivo applications the nucleotide sequences, antibodies and/or antigens of the invention are preferably administered as a component of an immunogenic composition comprising the nucleotide sequences and/or antigens of the invention in admixture with a pharmaceutically acceptable carrier. The immunogenic compositions of the invention are useful to stimulate an immune response against HIV-1 and may be used as one or more components of a prophylactic or therapeutic vaccine against HIV-1 for the prevention, amelioration or treatment of AIDS. The nucleic acids and vectors of the invention are particularly useful for providing genetic vaccines, i.e. vaccines for delivering the nucleic acids encoding the antibodies and/or antigens of the invention to a subject, such as a human, such that the antibodies and/or antigens are then expressed in the subject to elicit an immune response.

The compositions of the invention may be injectable suspensions, solutions, sprays, lyophilized powders, syrups, elixirs and the like. Any suitable form of composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

An immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.).

The immunogenic compositions of the invention can contain additional substances, such as wetting or emulsifying agents, buffering agents, or adjuvants to enhance the effectiveness of the vaccines (Remington's Pharmaceutical Sciences, 18th edition, Mack Publishing Company, (ed.) 1980).

Adjuvants may also be included. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71(3): 538-44; Ahmad-Nejad, P. et al (2002) Eur. J. Immunol. 32(7): 1958-68; poly IC or poly AU acids, polyarginine with or without CpG (also known in the art as IC31; see Schellack, C. et al (2003) Proceedings of the 34^{th} Annual Meeting of the German Society of Immunology; Lingnau, K. et al (2002) Vaccine 20(29-30): 3498-508), JuvaVax™ (U.S. Pat. No. 6,693,086), certain natural substances (e.g., wax D from Mycobacterium tuberculosis, substances found in Cornyebacterium parvum, Bordetella pertussis, or members of the genus Brucella), flagellin (Toll-like receptor 5 ligand; see McSorley, S. J. et al (2002) J. Immunol. 169(7): 3914-9), saponins such as Q521, Q517, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara®; U.S. Pat. Nos. 4,689,338; 5,238,944; Zuber, A. K. et al (2004) 22(13-14): 1791-8), and the CCR5 inhibitor CMPD167 (see Veazey, R. S. et al (2003) J. Exp. Med. 198: 1551-1562).

Aluminum hydroxide or phosphate (alum) are commonly used at 0.05 to 0.1% solution in phosphate buffered saline. Other adjuvants that can be used, especially with DNA vaccines, are cholera toxin, especially CTA1-DD/ISCOMs (see Mowat, A. M. et al (2001) J. Immunol. 167(6): 3398-405), polyphosphazenes (Allcock, H. R. (1998) App. Organometallic Chem. 12(10-11): 659-666; Payne, L. G. et al (1995) Pharm. Biotechnol. 6: 473-93), cytokines such as, but not limited to, IL-2, IL-4, GM-CSF, IL-12, IL-15 IGF-1, IFN-α, IFN-β, and IFN-γ (Boyer et al., (2002) J. Liposome Res. 121:137-142; WO01/095919), immunoregulatory proteins such as CD40L (ADX40; see, for example, WO03/063899), and the CD1a ligand of natural killer cells (also known as CRONY or α-galactosyl ceramide; see Green, T. D. et al, (2003) J. Virol. 77(3): 2046-2055), immunostimulatory fusion proteins such as IL-2 fused to the Fc fragment of immunoglobulins (Barouch et al., Science 290:486-492, 2000) and co-stimulatory molecules B7.1 and B7.2 (Boyer), all of which can be administered either as proteins or in the form of DNA, on the same expression vectors as those encoding the antigens of the invention or on separate expression vectors.

In an advantageous embodiment, the adjuvants may be lecithin combined with an acrylic polymer (Adjuplex-LAP), lecithin coated oil droplets in an oil-in-water emulsion (Adjuplex-LE) or lecithin and acrylic polymer in an oil-in-water emulsion (Adjuplex-LAO) (Advanced BioAdjuvants (ABA)).

The immunogenic compositions can be designed to introduce the nucleic acids or expression vectors to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulation can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

Suitable dosages of the nucleic acids and expression vectors of the invention (collectively, the immunogens) in the immunogenic composition of the invention can be readily determined by those of skill in the art. For example, the dosage of the immunogens can vary depending on the route of administration and the size of the subject. Suitable doses can be determined by those of skill in the art, for example by measuring the immune response of a subject, such as a laboratory animal, using conventional immunological techniques, and adjusting the dosages as appropriate. Such techniques for measuring the immune response of the subject include but are not limited to, chromium release assays, tetramer binding assays, IFN-y ELISPOT assays, IL-2 ELISPOT assays, intracellular cytokine assays, and other immunological detection assays, e.g., as detailed in the text “Antibodies: A Laboratory Manual” by Ed Harlow and David Lane.

When provided prophylactically, the immunogenic compositions of the invention are ideally administered to a subject in advance of HIV infection, or evidence of HIV infection, or in advance of any symptom due to AIDS, especially in high-risk subjects. The prophylactic administration of the immunogenic compositions can serve to provide protective immunity of a subject against HIV-1 infection or to prevent or attenuate the progression of AIDS in a subject already infected with HIV-1. When provided therapeutically, the immunogenic compositions can serve to ameliorate and treat AIDS symptoms and are advantageously used as soon after infection as possible, preferably before appearance of any symptoms of AIDS but may also be used at (or after) the onset of the disease symptoms.

The immunogenic compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. Further, delivery of DNA to animal tissue has been achieved by cationic liposomes (Watanabe et al., (1994) Mol. Reprod. Dev. 38:268-274; and WO 96/20013), direct injection of naked DNA into animal muscle tissue (Robinson et al., (1993) Vaccine 11:957-960; Hoffman et al., (1994) Vaccine 12: 1529-1533; Xiang et al., (1994) Virology 199: 132-140; Webster et al., (1994) Vaccine 12: 1495-1498; Davis et al., (1994) Vaccine 12: 1503-1509; and Davis et al., (1993) Hum. Mol. Gen. 2: 1847-1851), or intradermal injection of DNA using “gene gun” technology (Johnston et al., (1994) Meth. Cell Biol. 43:353-365). Alternatively, delivery routes can be oral, intranasal or by any other suitable route. Delivery also be accomplished via a mucosal surface such as the anal, vaginal or oral mucosa.

Immunization schedules (or regimens) are well known for animals (including humans) and can be readily determined for the particular subject and immunogenic composition. Hence, the immunogens can be administered one or more times to the subject. Preferably, there is a set time interval between separate administrations of the immunogenic composition. While this interval varies for every subject, typically it ranges from 10 days to several weeks, and is often 2, 4, 6 or 8 weeks. For humans, the interval is typically from 2 to 6 weeks. The immunization regimes typically have from 1 to 6 administrations of the immunogenic composition, but may have as few as one or two or four. The methods of inducing an immune response can also include administration of an adjuvant with the immunogens. In some instances, annual, biannual or other long interval (5-10 years) booster immunization can supplement the initial immunization protocol.

The present methods also include a variety of prime-boost regimens, for example DNA prime-Adenovirus boost regimens. In these methods, one or more priming immunizations are followed by one or more boosting immunizations. The actual immunogenic composition can be the same or different for each immunization and the type of immunogenic composition (e.g., containing protein or expression vector), the route, and formulation of the immunogens can also be varied. For example, if an expression vector is used for the priming and boosting steps, it can either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the invention to provide priming and boosting regimens.

A specific embodiment of the invention provides methods of inducing an immune response against HIV in a subject by administering an immunogenic composition of the invention, preferably comprising an adenovirus vector containing DNA encoding one or more of the epitopes of the invention, one or more times to a subject wherein the epitopes are expressed at a level sufficient to induce a specific immune response in the subject. Such immunizations can be repeated multiple times at time intervals of at least 2, 4 or 6 weeks (or more) in accordance with a desired immunization regime.

The immunogenic compositions of the invention can be administered alone, or can be co-administered, or sequentially administered, with other HIV immunogens and/or HIV immunogenic compositions, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

When used in combination, the other HIV immunogens can be administered at the same time or at different times as part of an overall immunization regime, e.g., as part of a prime-boost regimen or other immunization protocol. In an advantageous embodiment, the other HIV immunogen is env, preferably the HIV env trimer.

Many other HIV immunogens are known in the art, one such preferred immunogen is HIVA (described in WO 01/47955), which can be administered as a protein, on a plasmid (e.g., pTHr.HIVA) or in a viral vector (e.g., MVA.HIVA). Another such HIV immunogen is RENTA (described in PCT/US2004/037699), which can also be administered as a protein, on a plasmid (e.g., pTHr.RENTA) or in a viral vector (e.g., MVA.RENTA).

For example, one method of inducing an immune response against HIV in a human subject comprises administering at least one priming dose of an HIV immunogen and at least one boosting dose of an HIV immunogen, wherein the immunogen in each dose can be the same or different, provided that at least one of the immunogens is an epitope of the present invention, a nucleic acid encoding an epitope of the invention or an expression vector, preferably a VSV vector, encoding an epitope of the invention, and wherein the immunogens are administered in an amount or expressed at a level sufficient to induce an HIV-specific immune response in the subject. The HIV-specific immune response can include an HIV-specific T-cell immune response or an HIV-specific B-cell immune response. Such immunizations can be done at intervals, preferably of at least 2-6 or more weeks.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

The invention is further described by the following numbered paragraphs:

1. A method of screening for glycan-dependent self reactivities comprising immunoprecipitating a non-human immunodeficiency virus (HIV) protein from a media with a broadly neutralizing antibody.

2. The method of paragraph 1 wherein the broadly neutralizing antibody is selected from the group consisting of PGT151 and PGT121 and VRC06.

3. The method of paragraph 1 or 2 wherein the media is spent tissue culture (TC) supernatant.

4. The method of any one of paragraphs 1-3 wherein the broadly neutralizing antibody is PGT151.

5. The method of paragraph 4 wherein the non-HIV protein is galectin 3 binding protein (gal3BP).

6. A method of defining cross recognition comprising determining immuprecipitating putative unmutated ancestral antibodies (UAs) of a broadly neutralizing antibody and the non-HIV protein from any one of paragraphs 1-5, wherein a lack of immunoprecipitation suggests that the germline version of the antibody does not recognize the non-HIV protein. Therefore recognition likely evolved during the affinity maturation process in the germinal center (GC) reaction by somatic hypermutation and breaking of peripheral tolerance to now recognize the human self-protein.

7. The method of paragraph 6 wherein the broadly neutralizing antibody is PGT151.

8. The method of paragraph 6 or 7 wherein the non-HIV protein is gal3BP. 9. A method of eliciting trimer-specific and/or N-glycan-dependent broadly neutralizing antibodies in a patient in need thereof comprising administering the non-HIV protein of any one of paragraphs 1-8 to the patient.

10. The method of paragraph 9 wherein the non-HIV protein is modified.

11. The method of paragraph 9 or 10 wherein the non-HIV protein is arrayed on a particle.

12. The method of paragraph 11 wherein the arraying on particle is on an HPV particle or a liposome.

13. The method of any one of paragraphs 10-12 wherein the non-HIV protein is modified with heterologous T cell help as a monomer.

14. The method of any one of paragraphs 10-13 wherein the protein is modified by N/C His, Padre, TT peptide (P30), and/or free Cysteine.

15. The method of any one of paragraphs 9-14 further comprising heterologous cell help.

16. The method of paragraph 15 wherein the heterologous cell help is like from flu HA (13 residues or so, or multiple T helper epitopes) or PADRE (pan-DR epitopes) or the TT peptide by genetic fusion to the non-HIV self-protein, either at the C- or N-terminus, that will then be expressed from 293F cells as a recombinant fusion protein containing these so-called “promiscuous” T helper peptides (PADRE, TT, HA).

17. The method of any one of paragraphs 10-16 wherein the modified protein re-elicits broadly neutralizing antibody like monoclonal antibodies alone or in combination with Env trimers.

18. The method of any one of paragraphs 9-17 wherein the protein is gal3BP. 19. The method of any one of paragraphs 9-18 wherein the broadly neutralizing antibody is PGT151.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method of screening for glycan-dependent self reactivities comprising immunoprecipitating a non-human immunodeficiency virus (HIV) protein from a media with a broadly neutralizing antibody.

2. The method of claim 1 wherein the broadly neutralizing antibody is selected from the group consisting of PGT151 and PGT121 and VRC06.

3. The method of claim 1 wherein the media is spent tissue culture (TC) supernatant.

4. The method of claim 1 wherein the broadly neutralizing antibody is PGT151.

5. The method of claim 4 wherein the non-HIV protein is galectin 3 binding protein (gal3BP).

6. A method of defining cross recognition comprising determining immuprecipitating putative unmutated ancestral antibodies (UAs) of a broadly neutralizing antibody and the non-HIV protein from any one of claims 1-5, wherein a lack of immunoprecipitation suggests that the germline version of the antibody does not recognize the non-HIV protein. Therefore recognition likely evolved during the affinity maturation process in the germinal center (GC) reaction by somatic hypermutation and breaking of peripheral tolerance to now recognize the human self-protein.

7. The method of claim 6 wherein the broadly neutralizing antibody is PGT151.

8. The method of claim 6 wherein the non-HIV protein is gal3BP.

9. A method of eliciting trimer-specific and/or N-glycan-dependent broadly neutralizing antibodies in a patient in need thereof comprising administering the non-HIV protein of claim 1 to the patient.

10. The method of claim 9 wherein the non-HIV protein is modified.

11. The method of claim 9 wherein the non-HIV protein is arrayed on a particle.

12. The method of claim 11 wherein the arraying on particle is on an HPV particle or a liposome.

13. The method of claim 10 wherein the non-HIV protein is modified with heterologous T cell help as a monomer.

14. The method of claim 10 wherein the protein is modified by N/C His, Padre, TT peptide (P30), and/or free Cysteine.

15. The method of claim 9 further comprising heterologous cell help.

16. The method of claim 15 wherein the heterologous cell help is like from flu HA (13 residues or so, or multiple T helper epitopes) or PADRE (pan-DR epitopes) or the TT peptide by genetic fusion to the non-HIV self-protein, either at the C- or N-terminus, that will then be expressed from 293F cells as a recombinant fusion protein containing these so-called “promiscuous” T helper peptides (PADRE, TT, HA).

17. The method of claim 10 wherein the modified protein re-elicits broadly neutralizing antibody like monoclonal antibodies alone or in combination with Env trimers.

18. The method of claim 9 wherein the protein is gal3BP.

19. The method of claim 9 wherein the broadly neutralizing antibody is PGT151.


SEQUENCE LISTING (18 sequences)

SEQ ID NO: 1 - protein, 595 aa, Artificial Sequence
  Description of Artificial Sequence Synthetic polypeptide
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHH

SEQ ID NO: 2 - protein, 605 aa, Artificial Sequence
  Description of Artificial Sequence Synthetic polypeptide
MTPPRLFWVWLLVAGTQGHHHHHHGGGGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHH

SEQ ID NO: 3 - protein, 611 aa, Artificial Sequence
  Description of Artificial Sequence Synthetic polypeptide
MTPPRLFWVWLLVAGTQGHHHHHHGGSGAKFVAAWTLKAAAGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 4 - protein, 611 aa, Artificial Sequence
  Description of Artificial Sequence Synthetic polypeptide
MTPPRLFWVWLLVAGTQGAKFVAAWTLKAAAGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHH

SEQ ID NO: 5 - protein, 611 aa, Artificial Sequence
  Description of Artificial Sequence Synthetic polypeptide
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGSGAKFVAAWTLKAAAGGGGHHHHHH

SEQ ID NO: 6 - protein, 619 aa, Artificial Sequence
  Description of Artificial Sequence Synthetic polypeptide
MTPPRLFWVWLLVAGTQGFNNFTVSFWLRVPKVSASHLEGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHH

SEQ ID NO: 7 - protein, 619 aa, Artificial Sequence
  Description of Artificial Sequence Synthetic polypeptide
MTPPRLFWVWLLVAGTQGHHHHHHGGSGFNNFTVSFWLRVPKVSASHLEGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 8 - protein, 619 aa, Artificial Sequence
  Description of Artificial Sequence Synthetic polypeptide
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGSGFNNFTVSFWLRVPKVSASHLEGGGGHHHHHH

SEQ ID NO: 9 - protein, 596 aa, Artificial Sequence
  Description of Artificial Sequence Synthetic polypeptide
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHHC

SEQ ID NO: 10 - protein, 606 aa, Artificial Sequence
  Description of Artificial Sequence Synthetic polypeptide
MTPPRLFWVWLLVAGTQGCHHHHHHGGGGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHH

SEQ ID NO: 11 - protein, 612 aa, Artificial Sequence
  Description of Artificial Sequence Synthetic polypeptide
MTPPRLFWVWLLVAGTQGCHHHHHHGGSGAKFVAAWTLKAAAGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 12 - protein, 612 aa, Artificial Sequence
  Description of Artificial Sequence Synthetic polypeptide
MTPPRLFWVWLLVAGTQGAKFVAAWTLKAAAGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHHC

SEQ ID NO: 13 - protein, 612 aa, Artificial Sequence
  Description of Artificial Sequence Synthetic polypeptide
MTPPRLFWVWLLVAGTQGCAKFVAAWTLKAAAGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHH

SEQ ID NO: 14 - protein, 612 aa, Artificial Sequence
  Description of Artificial Sequence Synthetic polypeptide
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGSGAKFVAAWTLKAAAGGGGHHHHHHC

SEQ ID NO: 15 - protein, 620 aa, Artificial Sequence
  Description of Artificial Sequence Synthetic polypeptide
MTPPRLFWVWLLVAGTQGFNNFTVSFWLRVPKVSASHLEGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHHC

SEQ ID NO: 16 - protein, 620 aa, Artificial Sequence
  Description of Artificial Sequence Synthetic polypeptide
MTPPRLFWVWLLVAGTQGCFNNFTVSFWLRVPKVSASHLEGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGGGGHHHHHH

SEQ ID NO: 17 - protein, 620 aa, Artificial Sequence
  Description of Artificial Sequence Synthetic polypeptide
MTPPRLFWVWLLVAGTQGCHHHHHHGGSGFNNFTVSFWLRVPKVSASHLEGSGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 18 - protein, 620 aa, Artificial Sequence
  Description of Artificial Sequence Synthetic polypeptide
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDGSGFNNFTVSFWLRVPKVSASHLEGGGGHHHHHHC